SEQUENCE LISTING (41 sequences)

SEQ ID NO: 1 - DNA, 867 bp, Homo sapiens
atgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtggttggtgtcgtgggcggcctgctgggcagcctggtgctgctagtctgggtcctggccgtcatctgctcccgggccgcacgagggacaataggagccaggcgcaccggccagcccctgaaggaggacccctcagccgtgcctgtgttctctgtggactatggggagctggatttccagtggcgagagaagaccccggagccccccgtgccctgtgtccctgagcagacggagtatgccaccattgtctttcctagcggaatgggcacctcatcccccgcccgcaggggctcagctgacggccctcggagtgcccagccactgaggcctgaggatggacactgctcttggcccctctga

SEQ ID NO: 2 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 3 - DNA, 873 bp, Homo sapiens
atgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaa

SEQ ID NO: 4 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 5 - DNA, 531 bp, Homo sapiens
atgaggatatttgctgtctttatattcatgacctactggcatttgctgaacgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaa

SEQ ID NO: 6 - protein, 176 aa, Homo sapiens
MRIFAVFIFMTYWHLLNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 7 - DNA, 537 bp, Homo sapiens
atgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggagattag

SEQ ID NO: 8 - protein, 178 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGD

SEQ ID NO: 9 - DNA, 822 bp, Homo sapiens
atgatcttcctcctgctaatgttgagcctggaattgcagcttcaccagatagcagctttattcacagtgacagtccctaaggaactgtacataatagagcatggcagcaatgtgaccctggaatgcaactttgacactggaagtcatgtgaaccttggagcaataacagccagtttgcaaaaggtggaaaatgatacatccccacaccgtgaaagagccactttgctggaggagcagctgcccctagggaaggcctcgttccacatacctcaagtccaagtgagggacgaaggacagtaccaatgcataatcatctatggggtcgcctgggactacaagtacctgactctgaaagtcaaagcttcctacaggaaaataaacactcacatcctaaaggttccagaaacagatgaggtagagctcacctgccaggctacaggttatcctctggcagaagtatcctggccaaacgtcagcgttcctgccaacaccagccactccaggacccctgaaggcctctaccaggtcaccagtgttctgcgcctaaagccaccccctggcagaaacttcagctgtgtgttctggaatactcacgtgagggaacttactttggccagcattgaccttcaaagtcagatggaacccaggacccatccaacttggctgcttcacattttcatccccttctgcatcattgctttcattttcatagccacagtgatagccctaagaaaacaactctgtcaaaagctgtattcttcaaaagacacaacaaaaagacctgtcaccacaacaaagagggaagtgaacagtgctatctga

SEQ ID NO: 10 - protein, 273 aa, Homo sapiens
MIFLLLMLSLELQLHQIAALFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTWLLHIFIPFCIIAFIFIATVIALRKQLCQKLYSSKDTTKRPVTTTKREVNSAI

SEQ ID NO: 11 - DNA, 2502 bp, Homo sapiens
atggacgtcgtggaccctgacattttcaatagagacccccgggaccactatgacctgctacagcggctgggtggcggcacgtatggggaagtctttaaggctcgagacaaggtgtcaggggacctggtggcactgaagatggtgaagatggagcctgatgatgatgtctccacccttcagaaggaaatcctcatattgaaaacttgccggcacgccaacatcgtggcctaccatgggagttatctctggttgcagaaactctggatctgcatggaattctgtggggctggttctctccaggacatctaccaagtgacaggctccctgtcagagctccagattagctatgtctgccgggaagtgctccagggactggcctatttgcactcacagaagaagatacacagggacatcaagggagctaacatcctcatcaatgatgctggggaggtcagattggctgactttggcatctcggcccagattggggctacactggccagacgcctctctttcattgggacaccctactggatggctccggaagtggcagctgtggccctgaagggaggatacaatgagctgtgtgacatctggtccctgggcatcacggccatcgaactggccgagctacagccaccgctctttgatgtgcaccctctcagagttctcttcctcatgaccaagagtggctaccagcctccccgactgaaggaaaaaggcaaatggtcggctgccttccacaacttcatcaaagtcactctgactaagagtcccaagaaacgacccagcgccaccaagatgctcagtcatcaactggtatcccagcctgggctgaatcgaggcctgatcctggatcttcttgacaaactgaagaatcccgggaaaggaccctccattggggacattgaggatgaggagcccgagctaccccctgctatccctcggcggatcagatccacccaccgctccagctctctggggatcccagatgcagactgctgtcggcggcacatggagttcaggaagctccgaggaatggagaccagacccccagccaacaccgctcgcctacagcctcctcgagacctcaggagcagcagccccaggaagcaactgtcagagtcgtctgacgatgactatgacgacgtggacatccccacccctgcagaggacacacctcctccacttccccccaagcccaagttccgttctccatcagacgagggtcctgggagcatgggggatgatgggcagctgagcccgggggtgctggtccggtgtgccagtgggcccccaccaaacagcccccgtcctgggcctcccccatccaccagcagcccccacctcaccgcccattcagaaccctcactctggaacccaccctcccgggagcttgacaagcccccacttctgccccccaagaaggaaaagatgaagagaaagggatgtgcccttctcgtaaagttgttcaatggctgccccctccggatccacagcacggccgcctggacacatccctccaccaaggaccagcacctgctcctgggggcagaggaaggcatcttcatcctgaaccggaatgaccaggaggccacgctggaaatgctctttcctagccggactacgtgggtgtactccatcaacaacgttctcatgtctctctcaggaaagaccccccacctgtattctcatagcatccttggcctgctggaacggaaagagaccagagcaggaaaccccatcgctcacattagcccccaccgcctactggcaaggaagaacatggtttccaccaagatccaggacaccaaaggctgccgggcgtgctgtgtggcggagggtgcgagctctgggggcccgttcctgtgcggtgcattggagacgtccgttgtcctgcttcagtggtaccagcccatgaacaaattcctgcttgtccggcaggtgctgttcccactgccgacgcctctgtccgtgttcgcgctgctgaccgggccaggctctgagctgcccgctgtgtgcatcggcgtgagccccgggcggccggggaagtcggtgctcttccacacggtgcgctttggcgcgctctcttgctggctgggcgagatgagcaccgagcacaggggacccgtgcaggtgacccaggtagaggaagatatggtgatggtgttgatggatggctctgtgaagctggtgaccccggaggggtccccagtccggggacttcgcacacctgagatccccatgaccgaagcggtggaggccgtggctatggttggaggtcagcttcaggccttctggaagcatggagtgcaggtgtgggctctaggctcggatcagctgctacaggagctgagagaccctaccctcactttccgtctgcttggctcccccaggctggagtgcagtggcacgatctcgcctcactgcaacctcctcctcccaggttcaagcaattctcctgcctcagcctcccgagtagctgggattacaggcctgtag

SEQ ID NO: 12 - protein, 833 aa, Homo sapiens
MDVVDPDIFNRDPRDHYDLLQRLGGGTYGEVFKARDKVSGDLVALKMVKMEPDDDVSTLQKEILILKTCRHANIVAYHGSYLWLQKLWICMEFCGAGSLQDIYQVTGSLSELQISYVCREVLQGLAYLHSQKKIHRDIKGANILINDAGEVRLADFGISAQIGATLARRLSFIGTPYWMAPEVAAVALKGGYNELCDIWSLGITAIELAELQPPLFDVHPLRVLFLMTKSGYQPPRLKEKGKWSAAFHNFIKVTLTKSPKKRPSATKMLSHQLVSQPGLNRGLILDLLDKLKNPGKGPSIGDIEDEEPELPPAIPRRIRSTHRSSSLGIPDADCCRRHMEFRKLRGMETRPPANTARLQPPRDLRSSSPRKQLSESSDDDYDDVDIPTPAEDTPPPLPPKPKFRSPSDEGPGSMGDDGQLSPGVLVRCASGPPPNSPRPGPPPSTSSPHLTAHSEPSLWNPPSRELDKPPLLPPKKEKMKRKGCALLVKLFNGCPLRIHSTAAWTHPSTKDQHLLLGAEEGIFILNRNDQEATLEMLFPSRTTWVYSINNVLMSLSGKTPHLYSHSILGLLERKETRAGNPIAHISPHRLLARKNMVSTKIQDTKGCRACCVAEGASSGGPFLCGALETSVVLLQWYQPMNKFLLVRQVLFPLPTPLSVFALLTGPGSELPAVCIGVSPGRPGKSVLFHTVRFGALSCWLGEMSTEHRGPVQVTQVEEDMVMVLMDGSVKLVTPEGSPVRGLRTPEIPMTEAVEAVAMVGGQLQAFWKHGVQVWALGSDQLLQELRDPTLTFRLLGSPRLECSGTISPHCNLLLPGSSNSPASASRVAGITGL

SEQ ID NO: 13 - DNA, 2466 bp, Homo sapiens
atggacgtcgtggaccctgacattttcaatagagacccccgggaccactatgacctgctacagcggctgggtggcggcacgtatggggaagtctttaaggctcgagacaaggtgtcaggggacctggtggcactgaagatggtgaagatggagcctgatgatgatgtctccacccttcagaaggaaatcctcatattgaaaacttgccggcacgccaacatcgtggcctaccatgggagttatctctggttgcagaaactctggatctgcatggaattctgtggggctggttctctccaggacatctaccaagtgacaggctccctgtcagagctccagattagctatgtctgccgggaagtgctccagggactggcctatttgcactcacagaagaagatacacagggacatcaagggagctaacatcctcatcaatgatgctggggaggtcagattggctgactttggcatctcggcccagattggggctacactggccagacgcctctctttcattgggacaccctactggatggctccggaagtggcagctgtggccctgaagggaggatacaatgagctgtgtgacatctggtccctgggcatcacggccatcgaactggccgagctacagccaccgctctttgatgtgcaccctctcagagttctcttcctcatgaccaagagtggctaccagcctccccgactgaaggaaaaaggcaaatggtcggctgccttccacaacttcatcaaagtcactctgactaagagtcccaagaaacgacccagcgccaccaagatgctcagtcatcaactggtatcccagcctgggctgaatcgaggcctgatcctggatcttcttgacaaactgaagaatcccgggaaaggaccctccattggggacattgaggatgaggagcccgagctaccccctgctatccctcggcggatcagatccacccaccgctccagctctctggggatcccagatgcagactgctgtcggcggcacatggagttcaggaagctccgaggaatggagaccagacccccagccaacaccgctcgcctacagcctcctcgagacctcaggagcagcagccccaggaagcaactgtcagagtcgtctgacgatgactatgacgacgtggacatccccacccctgcagaggacacacctcctccacttccccccaagcccaagttccgttctccatcagacgagggtcctgggagcatgggggatgatgggcagctgagcccgggggtgctggtccggtgtgccagtgggcccccaccaaacagcccccgtcctgggcctcccccatccaccagcagcccccacctcaccgcccattcagaaccctcactctggaacccaccctcccgggagcttgacaagcccccacttctgccccccaagaaggaaaagatgaagagaaagggatgtgcccttctcgtaaagttgttcaatggctgccccctccggatccacagcacggccgcctggacacatccctccaccaaggaccagcacctgctcctgggggcagaggaaggcatcttcatcctgaaccggaatgaccaggaggccacgctggaaatgctctttcctagccggactacgtgggtgtactccatcaacaacgttctcatgtctctctcaggaaagaccccccacctgtattctcatagcatccttggcctgctggaacggaaagagaccagagcaggaaaccccatcgctcacattagcccccaccgcctactggcaaggaagaacatggtttccaccaagatccaggacaccaaaggctgccgggcgtgctgtgtggcggagggtgcgagctctgggggcccgttcctgtgcggtgcattggagacgtccgttgtcctgcttcagtggtaccagcccatgaacaaattcctgcttgtccggcaggtgctgttcccactgccgacgcctctgtccgtgttcgcgctgctgaccgggccaggctctgagctgcccgctgtgtgcatcggcgtgagccccgggcggccggggaagtcggtgctcttccacacggtgcgctttggcgcgctctcttgctggctgggcgagatgagcaccgagcacaggggacccgtgcaggtgacccaggtagaggaagatatggtgatggtgttgatggatggctctgtgaagctggtgaccccggaggggtccccagtccggggacttcgcacacctgagatccccatgaccgaagcggtggaggccgtggctatggttggaggtcagcttcaggccttctggaagcatggagtgcaggtgtgggctctaggctcggatcagctgctacaggagctgagagaccctaccctcactttccgtctgcttggctcccccaggcctgtagtggtggagacacgcccagtggatgatcctactgctcccagcaacctctacatccaggaatga

SEQ ID NO: 14 - protein, 821 aa, Homo sapiens
MDVVDPDIFNRDPRDHYDLLQRLGGGTYGEVFKARDKVSGDLVALKMVKMEPDDDVSTLQKEILILKTCRHANIVAYHGSYLWLQKLWICMEFCGAGSLQDIYQVTGSLSELQISYVCREVLQGLAYLHSQKKIHRDIKGANILINDAGEVRLADFGISAQIGATLARRLSFIGTPYWMAPEVAAVALKGGYNELCDIWSLGITAIELAELQPPLFDVHPLRVLFLMTKSGYQPPRLKEKGKWSAAFHNFIKVTLTKSPKKRPSATKMLSHQLVSQPGLNRGLILDLLDKLKNPGKGPSIGDIEDEEPELPPAIPRRIRSTHRSSSLGIPDADCCRRHMEFRKLRGMETRPPANTARLQPPRDLRSSSPRKQLSESSDDDYDDVDIPTPAEDTPPPLPPKPKFRSPSDEGPGSMGDDGQLSPGVLVRCASGPPPNSPRPGPPPSTSSPHLTAHSEPSLWNPPSRELDKPPLLPPKKEKMKRKGCALLVKLFNGCPLRIHSTAAWTHPSTKDQHLLLGAEEGIFILNRNDQEATLEMLFPSRTTWVYSINNVLMSLSGKTPHLYSHSILGLLERKETRAGNPIAHISPHRLLARKNMVSTKIQDTKGCRACCVAEGASSGGPFLCGALETSVVLLQWYQPMNKFLLVRQVLFPLPTPLSVFALLTGPGSELPAVCIGVSPGRPGKSVLFHTVRFGALSCWLGEMSTEHRGPVQVTQVEEDMVMVLMDGSVKLVTPEGSPVRGLRTPEIPMTEAVEAVAMVGGQLQAFWKHGVQVWALGSDQLLQELRDPTLTFRLLGSPRPVVVETRPVDDPTAPSNLYIQE

SEQ ID NO: 15 - protein, 25 aa, Artificial Sequence [sequence omitted]

SEQ ID NO: 16 - protein, 13 aa, Artificial Sequence
  Description of Artificial Sequence Synthetic peptide
WVRQAPGKGLEWV

SEQ ID NO: 17 - protein, 32 aa, Artificial Sequence
  Description of Artificial Sequence Synthetic polypeptide
RFTISADTSKNTAYLQMNSLRAEDTAVYYCAR

SEQ ID NO: 18 - protein, 11 aa, Artificial Sequence
  Description of Artificial Sequence Synthetic peptide
WGQGTLVTVSA

SEQ ID NO: 19 - protein, 23 aa, Artificial Sequence
  Description of Artificial Sequence Synthetic peptide
DIQMTQSPSSLSASVGDRVTITC

SEQ ID NO: 20 - protein, 15 aa, Artificial Sequence
  Description of Artificial Sequence Synthetic peptide
WYQQKPGKAPKLLIY

SEQ ID NO: 21 - protein, 32 aa, Artificial Sequence
  Description of Artificial Sequence Synthetic polypeptide
GVPSRFSGSGSGTDFTLTISSLQPEDFATYYC

SEQ ID NO: 22 - protein, 11 aa, Artificial Sequence
  Description of Artificial Sequence Synthetic peptide
FGQGTKVEIKR

SEQ ID NO: 23 - protein, 440 aa, Artificial Sequence
  Description of Artificial Sequence Synthetic polypeptide
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 24 - protein, 214 aa, Artificial Sequence
  Description of Artificial Sequence Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 118 aa, Artificial Sequence
  Description of Artificial Sequence Synthetic polypeptide
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSA

SEQ ID NO: 26 - protein, 108 aa, Artificial Sequence
  Description of Artificial Sequence Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 27 - protein, 118 aa, Artificial Sequence [sequence omitted]

SEQ ID NO: 28 - protein, 11 aa, Artificial Sequence
  Description of Artificial Sequence Synthetic peptide
WGQGTLVTVSA

SEQ ID NO: 29 - protein, 10 aa, Artificial Sequence
  Description of Artificial Sequence Synthetic peptide
GFTFSXSWIH

SEQ ID NO: 30 - protein, 18 aa, Artificial Sequence
  Description of Artificial Sequence Synthetic peptide
AWIXPYGGSXYYADSVKG

SEQ ID NO: 31 - protein, 9 aa, Artificial Sequence
  Description of Artificial Sequence Synthetic peptide
RHWPGGFDY

SEQ ID NO: 32 - protein, 11 aa, Artificial Sequence
  Description of Artificial Sequence Synthetic peptide
RASQXXXTXXA

SEQ ID NO: 33 - protein, 7 aa, Artificial Sequence
  Description of Artificial Sequence Synthetic peptide
SASXLXS

SEQ ID NO: 34 - protein, 9 aa, Artificial Sequence
  Description of Artificial Sequence Synthetic peptide
QQXXXXPXT

SEQ ID NO: 35 - protein, 10 aa, Artificial Sequence
  Description of Artificial Sequence Synthetic peptide
GFTFSDSWIH

SEQ ID NO: 36 - protein, 18 aa, Artificial Sequence
  Description of Artificial Sequence Synthetic peptide
AWISPYGGSTYYADSVKG

SEQ ID NO: 37 - protein, 11 aa, Artificial Sequence
  Description of Artificial Sequence Synthetic peptide
RASQDVSTAVA

SEQ ID NO: 38 - protein, 7 aa, Artificial Sequence
  Description of Artificial Sequence Synthetic peptide
SASFLYS

SEQ ID NO: 39 - protein, 9 aa, Artificial Sequence
  Description of Artificial Sequence Synthetic peptide
QQYLYHPAT

SEQ ID NO: 40 - protein, 448 aa, Artificial Sequence
  Description of Artificial Sequence Synthetic polypeptide
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 214 aa, Artificial Sequence
  Description of Artificial Sequence Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2016/0158360 A1
(43) Pub. Date: Jun. 9, 2016

(54) METHODS AND COMPOSITIONS FOR TREATING CANCER USING PD-1 AXIS ANTAGONISTS AND HPK1 ANTAGONISTS

(71) Applicant: Genentech, Inc., South San Francisco, CA (US)

(72) Inventors: Sairy Hernandez, Belmont, CA (US); Ira Mellman, San Francisco, CA (US); Jing Qing, San Francisco, CA (US); Deepak Sampath, San Francisco, CA (US)

(73) Assignee: Genentech, Inc., South San Francisco, CA (US)

(21) Appl. No.: 14/959,221

(22) Filed: Dec. 4, 2015

Related U.S. Application Data
(60) Provisional application No. 62/087,944, filed on Dec. 5, 2014.

(51) Int. Cl. A61K 39/395; A61K 45/06

(52) U.S. Cl. CPC A61K 39/39558; A61K 45/06; A61K 2039/505

(57) ABSTRACT

Compositions and methods for enhancing an immune response and treating cancer are provided. Compositions comprise PD-1 axis antagonists and HPK1 antagonists. PD-1 axis antagonists include PD-1 antagonists, PD-L1 antagonists, and PD-L2 antagonists. PD-1 axis antagonists can inhibit the binding of PD-L1 and/or PD-L2 to PD-1. HPK1 antagonists include compounds that inhibit the serine/threonine kinase activity of HPK1. Methods for enhancing an immune response or treating cancer comprise administering a PD-1 axis antagonist and a HPK1 antagonist, sequentially or simultaneously, to a subject in need thereof.

### RELATED APPLICATIONS

This application claims the benefit of Provisional Application No. 62/087,944 filed Dec. 5, 2014. All the teachings of the above-referenced application are incorporated herein by reference.

### SEQUENCE LISTING

The instant application contains a Sequence Listing which has been submitted electronically in ASCII format and is hereby incorporated by reference in its entirety. Said ASCII copy, created on Nov. 25, 2015, is named P32458-US_SL.txt and is 58,570 bytes in size.

### BACKGROUND OF THE INVENTION

The major treatment modalities used by oncologists to treat cancer are surgical resection, radiation therapy, and classical chemotherapeutic drugs. Unfortunately, surgical resection is not a viable option for many tumors or forms of cancers. Further, radiation therapy and chemotherapeutic drugs do not target only diseased cells and therefore, end up damaging healthy cells. Therapeutics that more specifically target tumor cells are being developed by taking advantage of tumor-specific expression of antigens or inappropriate overexpression or activation of specific proteins within tumor cells, but tumor cells are prone to mutation and can become resistant to drugs that specifically target tumor cells.

A new cancer treatment paradigm has emerged that harnesses the patient's own immune system to overcome immunoevasive strategies utilized by many cancers and to enhance anti-tumor immunity. One such strategy is to inhibit negative regulators of immune responses that normally function to maintain peripheral tolerance, allowing tumor antigens to be recognized as non-self entities.

### BRIEF SUMMARY OF THE INVENTION

Compositions comprising a (programmed death-1) PD-1 axis antagonist and a hematopoietic progenitor kinase 1 (HPK1) antagonist are provided herein. The PD-1 axis antagonist can bind to and antagonize PD-1 or either of its two ligands, PD-L1 or PD-L2, interfering with signal transduction downstream of PD-1 or preventing the binding of ligands to the PD-1 receptor. Such compositions find use in enhancing immune function in a subject, particularly antitumor immunity. Accordingly, the compositions comprising the two antagonists also find use in treating conditions where enhanced immunogenicity is desired, such as increasing tumor immunogenicity for the treatment of cancer.

### BRIEF DESCRIPTION OF THE FIGURES

FIG. 1 depicts the longer isoform of human HPK1, which comprises an amino-terminal kinase domain, four proline-rich (PR) motifs and a carboxy-terminal citron homology domain.

FIG. 2 demonstrates anti-tumor effects of HPK1 kinase inhibition and anti-PDL1 antibody. The comparative data show the differences of anti-PD-L1 antibody anti-tumor efficacy in HPK1 kinase-dead knock-in mice than in wild-type mice. FIG. 2 provides the average tumor volume of wild-type and HPK1.kd mice treated with a control antibody or an anti-PD-L1 antibody. Line graphs are representative of eighteen to twenty mice per group. Day values represent days post anti-PD-L1 antibody treatment start date.

FIG. 3 demonstrates anti-tumor effects of HPK1 kinase inhibition and anti-PD-1 antibody. The comparative data show the differences of anti-PD-1 antibody anti-tumor efficacy in HPK1 kinase-dead knock-in mice than in wild-type mice. FIG. 3 provides the average tumor volume of wild-type and HPK1.kd mice treated with a control antibody or an anti-PD-1 antibody. Line graphs are representative of fifteen mice per group. Day values represent days post anti-PD-1 antibody treatment start date.

### DETAILED DESCRIPTION OF THE INVENTION

### I. Definitions

The term “antibody” includes monoclonal antibodies (including full length antibodies which have an immunoglobulin Fc region), antibody compositions with polyepitopic specificity, multispecific antibodies (e.g., bispecific antibodies, diabodies, and single-chain molecules, as well as antibody fragments (e.g., Fab, F(ab′)₂, and Fv). The term “immunoglobulin” (Ig) is used interchangeably with “antibody” herein.

The basic 4-chain antibody unit is a heterotetrameric glycoprotein composed of two identical light (L) chains and two identical heavy (H) chains. An IgM antibody consists of 5 of the basic heterotetramer units along with an additional polypeptide called a J chain, and contains 10 antigen binding sites, while IgA antibodies comprise from 2-5 of the basic 4-chain units which can polymerize to form polyvalent assemblages in combination with the J chain. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (V_{H}) followed by three constant domains (C_{H}) for each of the α and γ chains and four C_{H} domains for μ and ε isotypes. Each L chain has at the N-terminus, a variable domain (V_{L}) followed by a constant domain at its other end. The V_{L} is aligned with the V_{H} and the C_{L} is aligned with the first constant domain of the heavy chain (C_{H}1). Particular amino acid residues are believed to form an interface between the light chain and heavy chain variable domains. The pairing of a V_{H} and V_{L} together forms a single antigen-binding site. For the structure and properties of the different classes of antibodies, see e.g., Basic and Clinical Immunology, 8th Edition, Daniel P. Sties, Abba I. Ten and Tristram G. Parsolw (eds), Appleton & Lange, Norwalk, Conn., 1994, page 71 and Chapter 6. The L chain from any vertebrate species can be assigned to one of two clearly distinct types, called kappa and lambda, based on the amino acid sequences of their constant domains. Depending on the amino acid sequence of the constant domain of their heavy chains (CH), immunoglobulins can be assigned to different classes or isotypes. There are five classes of immunoglobulins: IgA, IgD, IgE, IgG and IgM, having heavy chains designated α, δ, ε, γ and μ, respectively. The γ and α classes are further divided into subclasses on the basis of relatively minor differences in the CH sequence and function, e.g., humans express the following subclasses: IgG1, IgG2A, IgG2B, IgG3, IgG4, IgA1 and IgA2.

The “variable region” or “variable domain” of an antibody refers to the amino-terminal domains of the heavy or light chain of the antibody. The variable domains of the heavy chain and light chain may be referred to as “VH” and “VL”, respectively. These domains are generally the most variable parts of the antibody (relative to other antibodies of the same class) and contain the antigen binding sites.

The term “variable” refers to the fact that certain segments of the variable domains differ extensively in sequence among antibodies. The V domain mediates antigen binding and defines the specificity of a particular antibody for its particular antigen. However, the variability is not evenly distributed across the entire span of the variable domains. Instead, it is concentrated in three segments called hypervariable regions (HVRs) both in the light-chain and the heavy chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen binding site of antibodies (see Kabat et al., Sequences of Immunological Interest, Fifth Edition, National Institute of Health, Bethesda, Md. (1991)). The constant domains are not involved directly in the binding of antibody to an antigen, but exhibit various effector functions, such as participation of the antibody in antibody-dependent cellular toxicity.

The term “monoclonal antibody” as used herein refers to an antibody obtained from a population of substantially homogeneous antibodies, i.e., the individual antibodies comprising the population are identical except for possible naturally occurring mutations and/or post-translation modifications (e.g., isomerizations, amidations) that may be present in minor amounts. Monoclonal antibodies are highly specific, being directed against a single antigenic site. In contrast to polyclonal antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies of the presently disclosed compositions and methods may be made by a variety of techniques, including, for example, the hybridoma method (e.g., Kohler and Milstein., Nature, 256:495-97 (1975); Hongo et al., Hybridoma, 14 (3): 253-260 (1995), Harlow et al., Antibodies: A Laboratory Manual, (Cold Spring Harbor Laboratory Press, 2^{nd} ed. 1988); Hammerling et al., in: Monoclonal Antibodies and T-Cell Hybridomas 563-681 (Elsevier, N. Y., 1981)), recombinant DNA methods (see, e.g., U.S. Pat. No. 4,816,567), phage-display technologies (see, e.g., Clackson et al., Nature, 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; and 5,661,016; Marks et al., Bio/Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg and Huszar, Intern. Rev. Immunol. 13: 65-93 (1995).

The term “naked antibody” refers to an antibody that is not conjugated to a cytotoxic moiety or radiolabel.

The terms “full-length antibody,” “intact antibody” or “whole antibody” are used interchangeably to refer to an antibody in its substantially intact form, as opposed to an antibody fragment. Specifically, whole antibodies include those with heavy and light chains including an Fc region. The constant domains may be native sequence constant domains (e.g., human native sequence constant domains) or amino acid sequence variants thereof. In some cases, the intact antibody may have one or more effector functions.

An “antibody fragment” comprises a portion of an intact antibody, and in most cases, the antigen binding and/or the variable region of the intact antibody. Examples of antibody fragments include Fab, Fab′, F(ab′)₂ and Fv fragments; diabodies; linear antibodies (see U.S. Pat. No. 5,641,870, Example 2; Zapata et al., Protein Eng. 8(10): 1057-1062 [1995]); single-chain antibody molecules and multispecific antibodies formed from antibody fragments. Papain digestion of antibodies produces two identical antigen-binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (C_{H}1). Each Fab fragment is monovalent with respect to antigen binding, i.e., it has a single antigen-binding site. Pepsin treatment of an antibody yields a single large F(ab′)₂ fragment which roughly corresponds to two disulfide linked Fab fragments having different antigen-binding activity and is still capable of cross-linking antigen. Fab′ fragments differ from Fab fragments by having a few additional residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The Fc fragment comprises the carboxy-terminal portions of both H chains held together by disulfides. The effector functions of antibodies are determined by sequences in the Fc region, the region which is also recognized by Fc receptors (FcR) found on certain types of cells.

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three HVRs specific for an antigen) has the ability to recognize and bind antigen, although at a lower affinity than the entire binding site.

“Single-chain Fv” also abbreviated as “sFv” or “scFv” are antibody fragments that comprise the V_{H} and V_{L} antibody domains connected into a single polypeptide chain. In some cases, the sFv polypeptide further comprises a polypeptide linker between the V_{H} and V_{L} domains which enables the sFv to form the desired structure for antigen binding. For a review of the sFv, see Pluckthun in The Pharmacology of Monoclonal Antibodies, vol. 113, Rosenburg and Moore eds., Springer-Verlag, New York, pp. 269-315 (1994).

“Functional fragments” of the antibodies useful in the presently disclosed compositions and methods comprise a portion of an intact antibody, generally including the antigen binding or variable region of the intact antibody or the Fc region of an antibody which retains or has modified FcR binding capability. Examples of antibody fragments include linear antibody, single-chain antibody molecules and multispecific antibodies formed from antibody fragments.

The term “diabodies” refers to small antibody fragments prepared by constructing sFv fragments (see preceding paragraph) with short linkers (about 5-10) residues) between the V_{H} and V_{L} domains such that inter-chain but not intra-chain pairing of the V domains is achieved, thereby resulting in a bivalent fragment, i.e., a fragment having two antigen-binding sites. Bispecific diabodies are heterodimers of two “crossover” sFv fragments in which the V_{H} and V_{L} domains of the two antibodies are present on different polypeptide chains. Diabodies are described in greater detail in, for example, EP 404,097; WO 93/11161; Hollinger et al., Proc. Natl. Acad. Sci. USA 90: 6444-6448 (1993).

The monoclonal antibodies useful in the presently disclosed compositions and methods specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is(are) identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 (1984)). Chimeric antibodies of interest herein include PRIMATIZED® antibodies wherein the antigen-binding region of the antibody is derived from an antibody produced by, e.g., immunizing macaque monkeys with an antigen of interest. As used herein, “humanized antibody” is used as a subset of “chimeric antibodies.”

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from an HVR (hereinafter defined) of the recipient are replaced by residues from an HVR of a non-human species (donor antibody) such as mouse, rat, rabbit or non-human primate having the desired specificity, affinity, and/or capacity. In some instances, framework (“FR”) residues of the human immunoglobulin are replaced by corresponding non-human residues. Furthermore, humanized antibodies may comprise residues that are not found in the recipient antibody or in the donor antibody. These modifications may be made to further refine antibody performance, such as binding affinity. In general, a humanized antibody will comprise substantially all of at least one, and typically two, variable domains, in which all or substantially all of the hypervariable loops correspond to those of a non-human immunoglobulin sequence, and all or substantially all of the FR regions are those of a human immunoglobulin sequence, although the FR regions may include one or more individual FR residue substitutions that improve antibody performance, such as binding affinity, isomerization, immunogenicity, etc. The number of these amino acid substitutions in the FR are typically no more than 6 in the H chain, and in the L chain, no more than 3. The humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, for example, Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409.

A “human antibody” is an antibody that possesses an amino-acid sequence corresponding to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol., 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

The term “hypervariable region,” “HVR,” or “HV,” when used herein refers to the regions of an antibody variable domain which are hypervariable in sequence and/or form structurally defined loops. Generally, antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, e.g., Xu et al., Immunity 13:37-45 (2000); Johnson and Wu, in Methods in Molecular Biology 248:1-25 (Lo, ed., Human Press, Totowa, N. J., 2003). Indeed, naturally occurring camelid antibodies consisting of a heavy chain only are functional and stable in the absence of light chain. See, e.g., Hamers-Casterman et al., Nature 363:446-448 (1993); Sheriff et al., Nature Struct. Biol. 3:733-736 (1996).

A number of HVR delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk, J. Mol. Biol. 196:901-917 (1987)). The AbM HVRs represent a compromise between the Kabat HVRs and Chothia structural loops, and are used by Oxford Molecular's AbM antibody modeling software. The “contact” HVRs are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B (Kabat |
| | | | | numbering) |
| H1 | H31-H35 | H26-H35 | H26-H32 | H30-H35 (Chothia |
| | | | | numbering) |
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2) and 89-97 or 89-96 (L3) in the VL and 26-35 (H1), 50-65 or 49-65 (H2) and 93-102, 94-102, or 95-102 (H3) in the VH. The variable domain residues are numbered according to Kabat et al., supra, for each of these definitions.

The expression “variable-domain residue-numbering as in Kabat” or “amino-acid-position numbering as in Kabat,” and variations thereof, refers to the numbering system used for heavy-chain variable domains or light-chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy-chain variable domain may include a single amino acid insert (residue 52a according to Kabat) after residue 52 of H2 and inserted residues (e.g. residues 82a, 82b, and 82c, etc. according to Kabat) after heavy-chain FR residue 82. The Kabat numbering of residues may be determined for a given antibody by alignment at regions of homology of the sequence of the antibody with a “standard” Kabat numbered sequence.

“Framework” or “FR” residues are those variable-domain residues other than the HVR residues as herein defined.

A “human consensus framework” or “acceptor human framework” is a framework that represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, 5^{th} Ed. Public Health Service, National Institutes of Health, Bethesda, Md. (1991). Examples include for the VL, the subgroup may be subgroup kappa I, kappa II, kappa III or kappa IV as in Kabat et al., supra. Additionally, for the VH, the subgroup may be subgroup I, subgroup II, or subgroup III as in Kabat et al., supra. Alternatively, a human consensus framework can be derived from the above in which particular residues, such as when a human framework residue is selected based on its homology to the donor framework by aligning the donor framework sequence with a collection of various human framework sequences. An acceptor human framework “derived from” a human immunoglobulin framework or a human consensus framework may comprise the same amino acid sequence thereof, or it may contain pre-existing amino acid sequence changes. In some embodiments, the number of pre-existing amino acid changes are 10 or less, 9 or less, 8 or less, 7 or less, 6 or less, 5 or less, 4 or less, 3 or less, or 2 or less.

A “VH subgroup III consensus framework” comprises the consensus sequence obtained from the amino acid sequences in variable heavy subgroup III of Kabat et al., supra. In one embodiment, the VH subgroup III consensus framework amino acid sequence comprises at least a portion or all of each of the following sequences:

(HC-FR1)
(SEQ ID NO: 15)
EVQLVESGGGLVQPGGSLRLSCAAS,
(HC-FR2)
(SEQ ID NO: 16)
WVRQAPGKGLEWV,,
(HC-FR3, SEQ ID NO: 17)
RFTISADTSKNTAYLQMNSLRAEDTAVYYCAR,
(HC-FR4)
(SEQ ID NO: 18)
WGQGTLVTVSA,.

A “VL kappa I consensus framework” comprises the consensus sequence obtained from the amino acid sequences in variable light kappa subgroup I of Kabat et al., supra. In one embodiment, the VH subgroup I consensus framework amino acid sequence comprises at least a portion or all of each of the following sequences:

(LC-FR1)
(SEQ ID NO: 19)
DIQMTQSPSSLSASVGDRVTITC,
(LC-FR2)
(SEQ ID NO: 20)
WYQQKPGKAPKLLIY ,
(LC-FR3)
(SEQ ID NO: 21)
GVPSRFSGSGSGTDFTLTISSLQPEDFATYYC,
(LC-FR4)
(SEQ ID NO: 22)
FGQGTKVEIKR.

An “amino-acid modification” at a specified position, e.g. of the Fc region, refers to the substitution or deletion of the specified residue, or the insertion of at least one amino acid residue adjacent to the specified residue. Insertion “adjacent” to a specified residue means insertion within one to two residues thereof. The insertion may be N-terminal or C-terminal to the specified residue. In some embodiments, the amino acid modification is a substitution.

An “affinity-matured” antibody is one with one or more alterations in one or more HVRs thereof that result in an improvement in the affinity of the antibody for antigen, compared to a parent antibody that does not possess those alteration(s). In one embodiment, an affinity-matured antibody has nanomolar or even picomolar affinities for the target antigen. Affinity-matured antibodies are produced by procedures known in the art. For example, Marks et al., Bio/Technology 10:779-783 (1992) describes affinity maturation by VH- and VL-domain shuffling. Random mutagenesis of HVR and/or framework residues is described by, for example: Barbas et al. Proc Nat. Acad. Sci. USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al, J. Mol. Biol. 226:889-896 (1992).

As use herein, the term “specifically binds to” or is “specific for” refers to measurable and reproducible interactions such as binding between a target and an antibody, which is determinative of the presence of the target in the presence of a heterogeneous population of molecules including biological molecules. For example, an antibody that specifically binds to a target (which can be an epitope) is an antibody that binds this target with greater affinity, avidity, more readily, and/or with greater duration than it binds to other unrelated targets. In one embodiment, the extent of binding of an antibody to an unrelated target is less than about 10% of the binding of the antibody to the target as measured, e.g., by a radioimmunoassay (RIA). In certain embodiments, an antibody that specifically binds to a target has a dissociation constant (Kd) of ≦1 μM, ≦100 nM, ≦10 nM, ≦1 nM, or ≦0.1 nM. In certain embodiments, an antibody specifically binds to an epitope on a protein that is conserved among the protein from different species. In another embodiment, specific binding can include, but does not require exclusive binding.

By “specific antagonist” is intended an agent that reduces, inhibits, or otherwise diminishes the activity of a defined target greater than that of an unrelated target. For example, a HPK1 specific antagonist reduces at least one biological activity of HPK1 by an amount that is statistically greater than the inhibitory effect of the antagonist on any other protein (e.g., other serine/threonine kinases). In some embodiments, the IC₅₀ of the antagonist for the target is about 90%, 80%, 70%, 60%, 50%, 40%, 30%, 20%, 10%, 5%, 1%, 0.1%, 0.01%, 0.001% or less of the IC₅₀ of the antagonist for a non-target.

As used herein, the term “immunoadhesin” designates antibody-like molecules which combine the binding specificity of a heterologous protein (an “adhesin”) with the effector functions of immunoglobulin constant domains. Structurally, the immunoadhesins comprise a fusion of an amino acid sequence with the desired binding specificity which is other than the antigen recognition and binding site of an antibody (i.e., is “heterologous”), and an immunoglobulin constant domain sequence. The adhesin part of an immunoadhesin molecule typically is a contiguous amino acid sequence comprising at least the binding site of a receptor or a ligand. The immunoglobulin constant domain sequence in the immunoadhesin may be obtained from any immunoglobulin, such as IgG-1, IgG-2 (including IgG2A and IgG2B), IgG-3, or IgG-4 subtypes, IgA (including IgA-1 and IgA-2), IgE, IgD or IgM. The Ig fusions can include the substitution of a domain of a polypeptide or antibody described herein in the place of at least one variable region within an Ig molecule. In some embodiments, the immunoglobulin fusion includes the hinge, CH2 and CH3, or the hinge, CH1, CH2 and CH3 regions of an IgG1 molecule. For the production of immunoglobulin fusions, see also U.S. Pat. No. 5,428,130. Immunoadhesin combinations of Ig Fc and extracellular domains of cell surface receptors are sometimes termed soluble receptors.

A “fusion protein” and a “fusion polypeptide” refer to a polypeptide having two portions covalently linked together, where each of the portions is a polypeptide having a different property. The property may be a biological property, such as activity in vitro or in vivo. The property may also be a simple chemical or physical property, such as binding to a target molecule, catalysis of a reaction, etc. The two portions may be linked directly by a single peptide bond or through a peptide linker but are in reading frame with each other.

A “blocking” antibody or an “antagonist” antibody is one that inhibits or reduces a biological activity of the antigen it binds. In some embodiments, blocking antibodies or antagonist antibodies substantially or completely inhibit the biological activity of the antigen.

The term “Fc region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Suitable native-sequence Fc regions for use in the antibodies described herein include human IgG1, IgG2 (IgG2A, IgG2B), IgG3 and IgG4.

“Fc receptor” or “FcR” describes a receptor that binds to the Fc region of an antibody. In some embodiments, the FcR is a native sequence human FcR. Moreover, the FcR can be one which binds an IgG antibody (a gamma receptor) and includes receptors of the FcγRI, FcγRII, and FcγRIII subclasses, including allelic variants and alternatively spliced forms of these receptors, FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain Inhibiting receptor FcγRIIB contains an immunoreceptor tyrosine-based inhibition motif (ITIM) in its cytoplasmic domain. (see M. Daëron, Annu. Rev. Immunol. 15:203-234 (1997). FcRs are reviewed in Ravetch and Kinet, Annu. Rev. Immunol. 9: 457-92 (1991); Capel et al., Immunomethods 4: 25-34 (1994); and de Haas et al., J. Lab. Clin. Med. 126: 330-41 (1995). Other FcRs, including those to be identified in the future, are encompassed by the term “FcR” herein.

The term “Fc receptor” or “FcR” also includes the neonatal receptor, FcRn, which is responsible for the transfer of maternal IgGs to the fetus. Guyer et al., J . Immunol. 117: 587 (1976) and Kim et al., J. Immunol. 24: 249 (1994). Methods of measuring binding to FcRn are known (see, e.g., Ghetie and Ward, Immunol. Today 18: (12): 592-8 (1997); Ghetie et al., Nature Biotechnology 15 (7): 637-40 (1997); Hinton et al., J. Biol. Chem. 279 (8): 6213-6 (2004); WO 2004/92219 (Hinton et al.). Binding to FcRn in vivo and serum half-life of human FcRn high-affinity binding polypeptides can be assayed, e.g., in transgenic mice or transfected human cell lines expressing human FcRn, or in primates to which the polypeptides having a variant Fc region are administered. WO 2004/42072 (Presta) describes antibody variants with improved or diminished binding to FcRs. See also, e.g., Shields et al., J. Biol. Chem. 9(2): 6591-6604 (2001).

The term “intrabody” refers to an antibody that is capable of binding to an intracellular protein within a cell. Intrabodies are generally expressed within a cell via delivery of an expression cassette encoding the antibody, typically as a scFv, and comprising various localization signals to target the antibody to an intracellular compartment of interest (see Lo et al. (2008) Handb Exp Pharmacol 181:343-373, which is incorporated herein in its entirety). Methods of stabilizing intrabodies are known in the art and include, but are not limited to modifications of immunoglobulin VL domains that lead to hyperstability (Cohen (1998) Oncogene 17(19):2445-2456) or expression of the antibodies as a fusion protein to other stable intracellular proteins, such as maltose binding protein (Shaki-Loewenstein (2005) J Immunol Methods 303(1-2):19-39.

The phrase “substantially reduced,” or “substantially different,” as used herein, denotes a sufficiently high degree of difference between two numeric values (generally one associated with a molecule and the other associated with a reference/comparator molecule) such that one of skill in the art would consider the difference between the two values to be of statistical significance within the context of the biological characteristic measured by said values (e.g., Kd values). The difference between said two values is, for example, greater than about 10%, greater than about 20%, greater than about 30%, greater than about 40%, and/or greater than about 50% as a function of the value for the reference/comparator molecule.

The term “substantially similar” or “substantially the same,” as used herein, denotes a sufficiently high degree of similarity between two numeric values (for example, one associated with an antibody described herein and the other associated with a reference/comparator antibody), such that one of skill in the art would consider the difference between the two values to be of little or no biological and/or statistical significance within the context of the biological characteristic measured by said values (e.g., Kd values). The difference between said two values is, for example, less than about 50%, less than about 40%, less than about 30%, less than about 20%, and/or less than about 10% as a function of the reference/comparator value.

As used herein, the term “cytokine” refers generically to proteins released by one cell population that act on another cell as intercellular mediators or have an autocrine effect on the cells producing the proteins. Examples of such cytokines include lymphokines, monokines; interleukins (“ILs”) such as IL-1, IL-la, IL-2, IL-3, IL-4, IL-5, IL-6, IL-7, IL-8, IL-9, IL10, IL-11, IL-12, IL-13, IL-15, IL-17A-F, IL-18 to IL-29 (such as IL-23), IL-31, including PROLEUKIN® rIL-2; a tumor-necrosis factor such as TNF-α or TNF-β, TGF-β1-3; and other polypeptide factors including leukemia inhibitory factor (“LIF”), ciliary neurotrophic factor (“CNTF”), CNTF-like cytokine (“CLC”), cardiotrophin (“CT”), and kit ligand (“KL”).

As used herein, the term “chemokine” refers to soluble factors (e.g., cytokines) that have the ability to selectively induce chemotaxis and activation of leukocytes. They also trigger processes of angiogenesis, inflammation, wound healing, and tumorigenesis. A non-limiting example of a chemokine is IL-8, a human homolog of murine keratinocyte chemoattractant (KC).

### II. Compositions Comprising a PD-1 Axis Antagonist and/or a HPK1 Antagonist and Methods of Using the Same

Compositions comprising a PD-1 axis antagonist and/or a HPK1 antagonist and methods of using the same are provided herein. Data presented herein demonstrate that a combination of HPK1 inhibition and blockade of the PD-1 axis reduces the growth of tumor cells in more than an additive manner. Both PD-1, along with its ligands PD-L1 and PD-L2, and HPK1 function as negative regulators of T cell activation. HPK1 also negatively regulates B cells and inhibition of HPK1 results in enhanced antigen presentation by antigen presenting cells, such as dendritic cells. PD-L1 is overexpressed in many cancers and often concomitant overexpression of PD-1 occurs in tumor infiltrating T cells, resulting in attenuation of T cell activation and evasion of immune surveillance, which contributes to impaired antitumor immune responses. (Keir M E et al. (2008) Annu. Rev. Immunol. 26:677). While not being bound by any theory or mechanism of action, it is believed that simultaneously targeting both the PD-1 axis and HPK1 enhances antitumor immune responses in more than an additive manner, leading to reduction of tumor growth that is unexpected. In some embodiments, the result effect is greater than the expected or calculated additive effect of the individual components separately. Thus, compositions comprising a PD-1 axis antagonist and a HPK1 antagonist find surprisingly effective use in enhancing an immune response and in the treatment of cancer.

A. PD-1 Axis Antagonists

The programmed death-1 (PD-1) protein, also known as CD279 or SLEB2, is a type I transmembrane protein and member of the B7-CD28 family of T cell regulators. PD-1 polynucleotides and polypeptides are known in the art (Ishida et al. (1992) EMBO J 11(11):3887-3895, which is herein incorporated by reference in its entirety). Non-limiting examples of PD-1 polynucleotides and polypeptides comprise the human PD-1 polynucleotide as set forth in SEQ ID NO: 1 (nucleotides 69-935 of GenBank Accession No. NM_005018.2) and the encoded human PD-1 polypeptide of 288 amino acids (Accession No. NP_005009.2) as set forth in SEQ ID NO: 2.

For ease of reference, motifs of PD-1 polypeptides will be discussed as they relate to human PD-1, which consists of an extracellular domain (aa35-145) comprising an Ig-like V type domain (aa35-145), followed by the transmembrane domain (aa171-191), and the intracellular tail (192-288) with an immunoreceptor tyrosine-based inhibitory motif (ITIM) and immunoreceptor tyrosine-based switch motif (ITSM), the latter of which is essential for the inhibition of TCR signaling.

PD-1 is expressed by activated T cells, B cells, and myeloid cells. Further, the majority of tumor infiltrating T lymphocytes overexpress PD-1 relative to T lymphocytes in normal tissues and peripheral blood T lymphocytes (Ahmadzadeh et al. (2009) Blood 114(8):1537).

PD-1 has two known ligands, programmed death ligand 1 (PD-L1) and programmed death ligand 2 (PD-L2). PD-L1, also referred to as B7-H1, B7-4, CD274, and B7-H, is a cell-surface protein and member of the B7 family. PD-L1 polynucleotides and polypeptides are known in the art (Dong et al. (1999) Nat Med 5(12):1365-1369, which is herein incorporated by reference in its entirety). Non-limiting examples of PD-L1 polynucleotides and polypeptides comprise the human PD-L1 isoform 1 polynucleotide as set forth in SEQ ID NO: 3 (nucleotides 109-981 of GenBank Accession No. NM_014143.3) and the encoded human PD-L1 isoform 1 polypeptide of 290 amino acids (Accession No. NP_054862.1) as set forth in SEQ ID NO: 4; the human PD-L1 isoform 2 polynucleotide as set forth in SEQ ID NO: 5 (nucleotides 109-639 of GenBank Accession No. NM_001267706.1) and the encoded human PD-L1 isoform 2 polypeptide of 176 amino acids (Accession No. NP_001254635.1) as set forth in SEQ ID NO: 6; and the predicted human PD-L1 isoform 3 polynucleotide as set forth in SEQ ID NO: 7 (nucleotides 213-749 of GenBank Accession No. XM 006716759.1) and the encoded predicted human PD-L1 isoform 3 polypeptide of 178 amino acids (Accession No. XP_006716822.1) as set forth in SEQ ID NO: 8.

PD-L1 is a putative transmembrane protein with the putative extracellular domain of human PD-L1 isoform spanning amino acid residues 19-238, the helical transmembrane domain from aa239-259, and the putative cytoplasmic tail extending from aa260-290. Within the extracellular domain exists an Ig-like V type and an Ig-like C-type domain from aa19-127 and aa133-225, respectively.

PD-L1 is found on almost all types of lymphohematopoietic cells and is constitutively expressed by T cells, B cells, macrophages and dendritic cells and is thought to be the primary mediator of PD-1-dependent immunosuppression. PD-L1 is also expressed by some non-hemoatopoietic cells and is overexpressed in many cancers, wherein its overexpression is often associated with poor prognosis (Okazaki T et al., Intern. Immun. 2007 19(7):813) (Thompson R H et al., Cancer Res 2006, 66(7):3381).

Along with binding PD-1, PD-L1 has also been shown to bind to CD80 or B7-1, which inhibits T-cell activation and cytokine production.

The other known ligand for PD-1, PD-L2, also referred to as B7-DC, Btdc, and CD273, is a cell surface protein. PD-L2 polynucleotides and polypeptides are known in the art (Latchman et al. (2001) Nature Immunol 2: 261-268; and Tseng et al. (2001) J Exp Med 193: 839-845, each of which is herein incorporated by reference in its entirety). Non-limiting examples of PD-L2 polynucleotides and polypeptides comprise the human PD-L2 polynucleotide as set forth in SEQ ID NO: 9 (nucleotides 274-1095 of GenBank Accession No. NM_025239) and the encoded human PD-L2 polypeptide of 273 amino acids (Accession No. NP_079515) as set forth in SEQ ID NO: 10.

Like PD-L1, PD-L2 is a putative transmembrane protein with the putative extracellular domain of human PD-L2 spanning aa20-220, the putative transmembrane domain from aa221-241 and the putative cytoplasmic domain from aa242-273. The extracellular domain comprises a Ig-like V type domain from aa21-118 and a Ig-like C2-type domain from aa122-203.

PD-L2 is expressed by antigen presenting cells, including dendritic cells, with expression also found in other non-hematopoietic tissues.

As demonstrated herein, simultaneous HPK1 and PD-1 axis inhibition results in an unexpectedly effective anti-tumor response. Thus, compositions provided herein comprise a PD-1 axis antagonist and a HPK1 antagonist.

The term “PD-1 axis antagonist” refers to a molecule that inhibits the interaction of a PD-1 axis binding partner (i.e., PD-1, PD-L1, PD-L2) with either one or more of its binding partners, so as to remove T-cell dysfunction resulting from signaling on the PD-1 signaling axis—with a result being to restore or enhance T-cell function (e.g., proliferation, cytokine production, target cell killing). As used herein, a PD-1 axis antagonist includes a PD-1 antagonist, a PD-L1 antagonist and a PD-L2 antagonist.

The term “PD-1 antagonist” refers to a molecule that decreases, blocks, inhibits, abrogates or interferes with signal transduction resulting from the interaction of PD-1 with one or more of its binding partners, such as PD-L1, PD-L2. In some embodiments, the PD-1 antagonist is a molecule that inhibits the binding of PD-1 to its binding partners. In a specific aspect, the PD-1 antagonist inhibits the binding of PD-1 to PD-L1 and/or PD-L2. For example, PD-1 antagonists include anti-PD-1 antibodies, immunoadhesins, fusion proteins, oligopeptides and other molecules that decrease, block, inhibit, abrogate or interfere with signal transduction resulting from the interaction of PD-1 with PD-L1 and/or PD-L2. PD-1 antagonists include those antagonists that bind to PD-1 (also referred to herein as PD-1 binding antagonists) and molecules that reduce the expression of PD-1, such as the silencing elements described elsewhere herein.

In one embodiment, a PD-1 antagonist reduces the negative co-stimulatory signal mediated by or through PD-1 cell surface proteins expressed on T lymphocytes so as to enhance effector responses to antigen recognition.

In some embodiments, the PD-1 antagonist is an immunoadhesin (e.g., an immunoadhesin comprising an extracellular or PD-1 binding portion of PD-L1 or PD-L2 fused to a constant region (e.g., an Fc region of an immunoglobulin sequence). In some embodiments, the PD-1 antagonist is AMP-224. AMP-224, also known as B7-DCIg, is a PD-L2-Fc fusion soluble receptor described in WO2010/027827 and WO2011/066342.

In some embodiments, the PD-1 antagonist is an anti-PD-1 antibody (e.g., a human antibody, a humanized antibody, or a chimeric antibody). In some embodiments, the anti-PD-1 antibody is a monoclonal antibody. In some embodiments, the anti-PD-1 antibody is an antibody fragment selected from the group consisting of Fab, Fab′-SH, Fv, scFv, and (Fab′)₂ fragments. In some embodiments, the anti-PD-1 antibody is a humanized antibody. In some embodiments, the anti-PD-1 antibody is a human antibody.

In some embodiments, the anti-PD-1 antibody is selected from the group consisting of MDX-1106, Merck 3475 and CT-011.

In some embodiments, the anti-PD-1 antibody is MDX-1106 (described in WO2006/121168, which is incorporated herein in its entirety) or an antigen-binding fragment thereof. Alternative names for “MDX-1106” include MDX-1106-04, ONO-4538, BMS-936558, Nivolumab, and Opdivo®. In some embodiments, the anti-PD-1 antibody is Nivolumab (CAS Registry Number: 946414-94-4). In other embodiments, the anti-PD-1 antibody is an antibody that binds to an epitope capable of binding MDX-1106 or competes with MDX-1106 for binding to PD-1 in a competitive binding assay.

In a still further embodiment, the anti-PD-1 antibody useful as a PD-1 antagonist comprises a heavy chain comprising the heavy chain amino acid sequence from SEQ ID NO:23 and/or a light chain comprising the light chain amino acid sequence from SEQ ID NO:24. The anti-PD-1 antibody useful in the presently disclosed compositions and methods can also be an anti-PD-1 antibody comprising a heavy chain and/or a light chain sequence, wherein:

(a) the heavy chain sequence has at least 85%, at least 90%, at least 91%, at least 92%, at least 93%, at least 94%, at least 95%, at least 96%, at least 97%, at least 98%, at least 99% or 100% sequence identity to the heavy chain sequence:

(SEQ ID NO: 23)
QVQLVESGGGVVQPGRSLRLDCKASGITFSNSGMHWVRQAPGKGLEW
VAVIWYDGSKRYYADSVKGRFTISRDNSKNTLFLQMNSLRAEDTAVY
YCATNDDYWGQGTLVTVSSASTKGPSVFPLAPCSRSTSESTAALGCL
VKDYFPEPVTVSWNSGALTSGVHTFPAVLQSSGLYSLSSVVTVPSSS
LGTKTYTCNVDHKPSNTKVDKRVESKYGPPCPPCPAPEFLGGPSVFL
FPPKPKDTLMISRTPEVTCVVVDVSQEDPEVQFNWYVDGVEVHNAKT
KPREEQFNSTYRVVSVLTVLHQDWLNGKEYKCKVSNKGLPSSIEKTI
SKAKGQPREPQVYTLPPSQEEMTKNQVSLTCLVKGFYPSDIAVEWES
NGQPENNYKTTPPVLDSDGSFFLYSRLTVDKSRWQEGNVFSCSVMHE
ALHNHYTQKSLSLSLGK,

or

(b) the light chain sequence has at least 85%, at least 90%, at least 91%, at least 92%, at least 93%, at least 94%, at least 95%, at least 96%, at least 97%, at least 98%, at least 99% or 100% sequence identity to the light chain sequence:

(SEQ ID NO: 24)
EIVLTQSPATLSLSPGERATLSCRASQSVSSYLAWYQQKPGQAPRLL
IYDASNRATGIPARFSGSGSGTDFTLTISSLEPEDFAVYYCQQSSNW
PRTFGQGTKVEIKRTVAAPSVFIFPPSDEQLKSGTASVVCLLNNFYP
REAKVQWKVDNALQSGNSQESVTEQDSKDSTYSLSSTLTLSKADYEK
HKVYACEVTHQGLSSPVTKSFNRGEC.

In other embodiments, the anti-PD-1 antibody is Merck 3475, also known as MK-3475, SCH-900475, pembrolizumab, lambrolizumab, and Keytruda® with CAS Registry Number: 1374853-91-4 (and described in WO2009/114335 and U.S. Pat. No. 8,354,509, each of which is herein incorporated by reference in its entirety), or an antigen-binding fragment thereof. In other embodiments, the anti-PD-1 antibody is an antibody that binds to an epitope capable of binding Merck 3475 or competes with Merck 3475 for binding to PD-1 in a competitive binding assay.

In still other embodiments, the anti-PD-1 antibody useful in the presently disclosed compositions and methods is CT-011, also known as hBAT, hBAT-1, or pidilizumab (and described in WO2009/101611, which is herein incorporated by reference in its entirety), or an antigen-binding fragment thereof. In other embodiments, the anti-PD-1 antibody is an antibody that binds to an epitope capable of binding CT-011 or competes with CT-011 for binding to PD-1 in a competitive binding assay.

The anti-PD-1 antibody can comprise a human or murine constant region. In some embodiments, the human constant region is selected from the group consisting of IgG1, IgG2, IgG2, IgG3, IgG4. In some of these embodiments, the human constant region is IgG1. In other embodiments, the murine constant region is selected from the group consisting of IgG1, IgG2A, IgG2B, IgG3. In some of these embodiments, the murine constant region is IgG2A.

In some embodiments, the anti-PD-1 antibody has reduced or minimal effector function. In some of these embodiments, the minimal effector function results from an “effector-less Fc mutation” or aglycosylation. In some embodiments, the effector-less Fc mutation is an N297A or D265A/N297A substitution in the constant region.

The PD-1 axis antagonist useful in the presently disclosed compositions and methods can comprise a PD-L1 antagonist. The term “PD-L1 antagonist” refers to a molecule that decreases, blocks, inhibits, abrogates or interferes with signal transduction resulting from the interaction of PD-L1 with either one or more of its binding partners, such as PD-1, B7-1. In some embodiments, a PD-L1 antagonist is a molecule that inhibits the binding of PD-L1 to its binding partners. In a specific aspect, the PD-L1 antagonist inhibits binding of PD-L1 to PD-1 and/or B7-1.

In some embodiments, the PD-L1 antagonists include anti-PD-L1 antibodies, immunoadhesins, fusion proteins, oligopeptides and other molecules that decrease, block, inhibit, abrogate or interfere with signal transduction resulting from the interaction of PD-L1 with one or more of its binding partners, such as PD-1, B7-1. PD-L1 antagonists include molecules that bind to PD-L1 (also referred to herein as PD-L1 binding antagonists) and molecules that reduce the expression of PD-L1, such as the silencing elements described elsewhere herein.

In one embodiment, a PD-L1 antagonist reduces the negative co-stimulatory signal mediated by or through PD-1 cell surface proteins expressed on T lymphocytes so as to enhance effector responses to antigen recognition.

In some embodiments, the PD-L1 antagonist is an immunoadhesin, such as a polypeptide that comprises the extracellular or PD-L1 binding portion of PD-1, fused to a constant domain of an immunoglobulin sequence (e.g., Fc).

In some embodiments, the PD-L1 antagonist is an anti-PD-L1 antibody. In some embodiments, the anti-PD-L1 antibody is a monoclonal antibody. In some embodiments, the anti-PD-L1 antibody is an antibody fragment selected from the group consisting of Fab, Fab′-SH, Fv, scFv, and (Fab′)₂ fragments. In some embodiments, the anti-PD-L1 antibody is a humanized antibody. In some embodiments, the anti-PD-L1 antibody is a human antibody.

Non-limiting examples of anti-PD-L1 antibodies useful in the presently disclosed compositions and methods, and methods for making thereof are described in PCT patent application WO 2010/077634 A1, which is incorporated herein by reference. In some embodiments, the anti-PD-L1 antibody is selected from the group consisting of YW243.55.S70, MPDL3280A, MDX-1105, and MEDI4736.

In some embodiments, the anti-PD-L1 is the MDX-1105 antibody, also known as BMS-936559, (which is described in WO2007/005874, herein incorporated by reference in its entirety) or an antigen-binding fragment thereof. In still other embodiments, the anti-PD-L1 antibody is an antibody that binds to an epitope capable of binding MDX-1105 or competes with MDX-1105 for binding to PD-L1 in a competitive binding assay.

In other embodiments, the anti-PD-L1 antibody is MEDI4736 (which is described in WO2011/066389 and US2013/034559, each of which is herein incorporated by reference in its entirety) or an antigen-binding fragment thereof. In still other embodiments, the anti-PD-L1 antibody is an antibody that binds to an epitope capable of binding MEDI4736 or competes with MEDI4736 for binding to PD-L1 in a competitive binding assay.

In one embodiment, the anti-PD-L1 antibody contains a heavy chain variable region polypeptide comprising an HVR-H1, HVR-H2 and HVR-H3 sequence, wherein:
(a) the HVR-H1 sequence is GFTFSX₁SWIH (SEQ ID NO:29);
(b) the HVR-H2 sequence is AWIX₂PYGGSX₃YYADSVKG (SEQ ID NO:30);
(c) the HVR-H3 sequence is RHWPGGFDY (SEQ ID NO:31);
further wherein: X₁ is D or G; X₂ is S or L; X₃ is T or S.

In one specific aspect, X₁ is D; X₂ is S and X₃ is T. In another aspect, the polypeptide further comprises variable region heavy chain framework sequences juxtaposed between the HVRs according to the formula: (HC-FR1)-(HVR-H1)-(HC-FR2)-(HVR-H2)-(HC-FR3)-(HVR-H3)-(HC-FR4). In yet another aspect, the framework sequences are derived from human consensus framework sequences. In a further aspect, the framework sequences are VH subgroup III consensus framework. In a still further aspect, at least one of the framework sequences is the following:

HC-FR1 is
(SEQ ID NO: 15)
EVQLVESGGGLVQPGGSLRLSCAAS
HC-FR2 is
(SEQ ID NO: 16)
WVRQAPGKGLEWV
HC-FR3 is
(SEQ ID NO: 17)
RFTISADTSKNTAYLQMNSLRAEDTAVYYCAR
HC-FR4 is
(SEQ ID NO: 18)
WGQGTLVTVSA.

In a still further aspect, the heavy chain polypeptide is further combined with a variable region light chain comprising an HVR-L1, HVR-L2 and HVR-L3, wherein:

(a) the HVR-L1 sequence is RASQX₄X₅X₆TX₇X₈A (SEQ ID NO:32);

(b) the HVR-L2 sequence is SASX₉LX₁₀S, (SEQ ID NO:33);

(c) the HVR-L3 sequence is QQX₁₁X₁₂X₁₃X₁₄PX₁₅T (SEQ ID NO:34);
further wherein: X₄ is D or V; X₅ is V or I; X₆ is S or N; X₇ is A or F; X₈ is V or L; X₉ is F or T; X₁₀ is Y or A; X₁₁ is Y, G, F, or S; X₁₂ is L, Y, F or W; X₁₃ is Y, N, A, T, G, F or I; X₁₄ is H, V, P, T or I; X₁₅ is A, W, R, P or T.

In a still further aspect, X₄ is D; X₅ is V; X₆ is S; X₇ is A; X₈ is V; X₉ is F; X₁₀ is Y; X₁₁ is Y; X₁₂ is L; X₁₃ is Y; X₁₄ is H; X₁₅ is A. In a still further aspect, the light chain further comprises variable region light chain framework sequences juxtaposed between the HVRs according to the formula: (LC-FR1)-(HVR-L1)-(LC-FR2)-(HVR-L2)-(LC-FR3)-(HVR-L3)-(LC-FR4). In a still further aspect, the framework sequences are derived from human consensus framework sequences. In a still further aspect, the framework sequences are VL kappa I consensus framework. In a still further aspect, at least one of the framework sequences is the following:

(SEQ ID NO: 19)
LC-FR1 is DIQMTQSPSSLSASVGDRVTITC
(SEQ ID NO: 20)
LC-FR2 is WYQQKPGKAPKLLIY
(SEQ ID NO: 21)
LC-FR3 is GVPSRFSGSGSGTDFTLTISSLQPEDFATYYC
(SEQ ID NO: 22)
LC-FR4 is FGQGTKVEIKR.

In another embodiment, the anti-PD-L1 antibody useful in the presently disclosed compositions and methods comprise a heavy chain and a light chain variable region sequence, wherein:
(a) the heavy chain comprises an HVR-H1, HVR-H2 and HVR-H3, wherein further:
   (i) the HVR-H1 sequence is GFTFSX₁SWIH (SEQ ID NO:29)
   (ii) the HVR-H2 sequence is AWIX₂PYGGSX₃YYADSVKG (SEQ ID NO:30)
   (iii) the HVR-H3 sequence is RHWPGGFDY, and (SEQ ID NO:31)
(b) the light chain comprises and HVR-L1, HVR-L2 and HVR-L3, wherein further:
   (i) the HVR-L1 sequence is RASQX₄X₅X₆TX₇X₈A (SEQ ID NO:32)
   (ii) the HVR-L2 sequence is SASX₉LX₁₀S; and (SEQ ID NO:33)
   (iii) the HVR-L3 sequence is QQX₁₁X₁₂X₁₃X₁₄PX₁₅T; (SEQ ID NO:34)
Further wherein: X₁ is D or G; X₂ is S or L; X₃ is T or S; X₄ is D or V; X₅ is V or I; X₆ is S or N; X₇ is A or F; X₈ is V or L; X₉ is F or T; X₁₀ is Y or A; Xᵢᵢ is Y, G, F, or S; X₁₂ is L, Y, F or W; X₁₃ is Y, N, A, T, G, F or I; X₁₄ is H, V, P, T or I; X₁₅ is A, W, R, P or T.

In a specific aspect, X₁ is D; X₂ is S and X₃ is T. In another aspect, X₄ is D; X₅ is V; X₆ is S; X₇ is A; X₈ is V; X₉ is F; X₁₀ is Y; X₁₁ is Y; X₁₂ is L; X₁₃ is Y; X₁₄ is H; X₁₅ is A. In yet another aspect, X₁ is D; X₂ is S and X₃ is T, X₄ is D; X₅ is V; X₆ is S; X₇ is A; X₈ is V; X₉ is F; X₁₀ is Y; X₁₁ is Y; X₁₂ is L; X₁₃ is Y; X₁₄ is H and X₁₅ is A.

In a further aspect, the heavy chain variable region comprises one or more framework sequences juxtaposed between the HVRs as: (HC-FR1)-(HVR-H1)-(HC-FR2)-(HVR-H2)-(HC-FR3)-(HVR-H3)-(HC-FR4), and the light chain variable regions comprises one or more framework sequences juxtaposed between the HVRs as: (LC-FR1)-(HVR-L1)-(LC-FR2)-(HVR-L2)-(LC-FR3)-(HVR-L3)-(LC-FR4). In a still further aspect, the framework sequences are derived from human consensus framework sequences. In a still further aspect, the heavy chain framework sequences are derived from a Kabat subgroup I, II, or III sequence. In a still further aspect, the heavy chain framework sequence is a VH subgroup III consensus framework. In a still further aspect, one or more of the heavy chain framework sequences is the following:

(SEQ ID NO: 15)
HC-FR1 EVQLVESGGGLVQPGGSLRLSCAAS
(SEQ ID NO: 16)
HC-FR2 WVRQAPGKGLEWV
(SEQ ID NO: 17)
HC-FR3 RFTISADTSKNTAYLQMNSLRAEDTAVYYCAR
(SEQ ID NO: 18)
HC-FR4 WGQGTLVTVSA.

In a still further aspect, the light chain framework sequences are derived from a Kabat kappa I, II, II or IV subgroup sequence. In a still further aspect, the light chain framework sequences are VL kappa I consensus framework. In a still further aspect, one or more of the light chain framework sequences is the following:

(SEQ ID NO: 19)
LC-FR1 DIQMTQSPSSLSASVGDRVTITC
(SEQ ID NO: 20)
LC-FR2 WYQQKPGKAPKLLIY
(SEQ ID NO: 21)
LC-FR3 GVPSRFSGSGSGTDFTLTISSLQPEDFATYYC
(SEQ ID NO: 22)
LC-FR4 FGQGTKVEIKR.

The anti-PD-L1 antibody further can comprise a human or murine constant region. In some embodiments, the human constant region is selected from the group consisting of IgG1, IgG2, IgG2, IgG3, IgG4. In some of these embodiments, the human constant region is IgG1. In other embodiments, the murine constant region is selected from the group consisting of IgG1, IgG2A, IgG2B, IgG3. In some of these embodiments, the murine constant region is IgG2A.

In some embodiments, the anti-PD-L1 antibody has reduced or minimal effector function. In some of these embodiments, the minimal effector function results from an “effector-less Fc mutation” or aglycosylation. In some embodiments, the effector-less Fc mutation is an N297A or D265A/N297A substitution in the constant region.

In yet another embodiment, the anti-PD-L1 antibody useful in the presently disclosed compositions and methods comprises a heavy chain and a light chain variable region sequence, wherein:
(a) the heavy chain further comprises an HVR-H1, HVR-H2 and an HVR-H3 sequence having at least 85% sequence identity to GFTFSDSWIH (SEQ ID NO:35), AWISPYGGSTYYADSVKG (SEQ ID NO:36) and RHWPGGFDY (SEQ ID NO:31), respectively, or
(b) the light chain further comprises an HVR-L1, HVR-L2 and an HVR-L3 sequence having at least 85% sequence identity to RASQDVSTAVA (SEQ ID NO:37), SASFLYS (SEQ ID NO:38) and QQYLYHPAT (SEQ ID NO:39), respectively.

In a specific aspect, the sequence identity is 86%, 87%, 88%, 89%, 90%, 91%, 92%, 93%, 94%, 95%, 96%, 97%, 98%, 99% or 100%. In another aspect, the heavy chain variable region comprises one or more framework sequences juxtaposed between the HVRs as: (HC-FR1)-(HVR-H1)-(HC-FR2)-(HVR-H2)-(HC-FR3)-(HVR-H3)-(HC-FR4), and the light chain variable regions comprises one or more framework sequences juxtaposed between the HVRs as: (LC-FR1)-(HVR-L1)-(LC-FR2)-(HVR-L2)-(LC-FR3)-(HVR-L3)-(LC-FR4). In yet another aspect, the framework sequences are derived from human consensus framework sequences. In a still further aspect, the heavy chain framework sequences are derived from a Kabat subgroup I, II, or III sequence. In a still further aspect, the heavy chain framework sequence is a VH subgroup III consensus framework. In a still further aspect, one or more of the heavy chain framework sequences is the following:

(SEQ ID NO: 15)
HC-FR1 EVQLVESGGGLVQPGGSLRLSCAAS
(SEQ ID NO: 16)
HC-FR2 WVRQAPGKGLEWV
(SEQ ID NO: 17)
HC-FR3 RFTISADTSKNTAYLQMNSLRAEDTAVYYCAR
(SEQ ID NO: 18)
HC-FR4 WGQGTLVTVSA.

In a still further aspect, the light chain framework sequences are derived from a Kabat kappa I, II, II or IV subgroup sequence. In a still further aspect, the light chain framework sequences are VL kappa I consensus framework. In a still further aspect, one or more of the light chain framework sequences is the following:

(SEQ ID NO: 19)
LC-FR1 DIQMTQSPSSLSASVGDRVTITC
(SEQ ID NO: 20)
LC-FR2 WYQQKPGKAPKLLIY
(SEQ ID NO: 21)
LC-FR3 GVPSRFSGSGSGTDFTLTISSLQPEDFATYYC
(SEQ ID NO: 22)
LC-FR4 FGQGTKVEIKR.

The anti-PD-L1 antibody further can comprise a human or murine constant region. In some embodiments, the human constant region is selected from the group consisting of IgG1, IgG2, IgG2, IgG3, IgG4. In some of these embodiments, the human constant region is IgG1. In other embodiments, the murine constant region is selected from the group consisting of IgG1, IgG2A, IgG2B, IgG3. In some of these embodiments, the murine constant region if IgG2A.

In some embodiments, the anti-PD-L1 antibody has reduced or minimal effector function. In some of these embodiments, the minimal effector function results from an “effector-less Fc mutation” or aglycosylation. In some embodiments, the effector-less Fc mutation is an N297A or D265A/N297A substitution in the constant region.

Antibody YW243.55.S70 (heavy and light chain variable region sequences shown in SEQ ID NOs. 25 and 26, respectively) is an anti-PD-L1 described in WO 2010/077634 A1. In some embodiments, the anti-PD-L1 antibody is the YW243.55.570 antibody or an antigen-binding fragment thereof. In other embodiments, the anti-PD-L1 useful in the presently disclosed compositions and methods comprises an antibody that binds to an epitope capable of binding YW243.55S70 or competes with YW243.55.S70 for binding to PD-L1 in a competitive binding assay.

In a still further embodiment, the anti-PD-L1 antibody comprises a heavy chain and a light chain variable region sequence, wherein:

(a) the heavy chain variable region sequence has at least 85% sequence identity to the heavy chain variable region sequence:

(SEQ ID NO: 25)
EVQLVESGGGLVQPGGSLRLSCAASGFTFSDSWIHWVRQAPGKGLEWVAW
ISPYGGSTYYADSVKGRFTISADTSKNTAYLQMNSLRAEDTAVYYCARRH
WPGGFDYWGQGTLVTVSA,

or

(b) the light chain variable region sequence has at least 85% sequence identity to the light chain variable region sequence:

(SEQ ID NO: 26)
DIQMTQSPSSLSASVGDRVTITCRASQDVSTAVAWYQQKPGKAPKLLIY
SASFLYSGVPSRFSGSGSGTDFTLTISSLQPEDFATYYCQQYLYHPATF
GQGTKVEIKR.

In a specific aspect, the sequence identity is 86%, 87%, 88%, 89%, 90%, 91%, 92%, 93%, 94%, 95%, 96%, 97%, 98%, 99% or 100%. In another aspect, the heavy chain variable region comprises one or more framework sequences juxtaposed between the HVRs as: (HC-FR1)-(HVR-H1)-(HC-FR2)-(HVR-H2)-(HC-FR3)-(HVR-H3)-(HC-FR4), and the light chain variable regions comprises one or more framework sequences juxtaposed between the HVRs as: (LC-FR1)-(HVR-L1)-(LC-FR2)-(HVR-L2)-(LC-FR3)-(HVR-L3)-(LC-FR4). In yet another aspect, the framework sequences are derived from human consensus framework sequences. In a further aspect, the heavy chain framework sequences are derived from a Kabat subgroup I, II, or III sequence. In a still further aspect, the heavy chain framework sequence is a VH subgroup III consensus framework. In a still further aspect, one or more of the heavy chain framework sequences is the following:

(SEQ ID NO: 15)
HC-FR1 EVQLVESGGGLVQPGGSLRLSCAAS
(SEQ ID NO: 16)
HC-FR2 WVRQAPGKGLEWV
(SEQ ID NO: 17)
HC-FR3 RFTISADTSKNTAYLQMNSLRAEDTAVYYCAR
(SEQ ID NO: 18)
HC-FR4 WGQGTLVTVSA.

In a still further aspect, the light chain framework sequences are derived from a Kabat kappa I, II, II or IV subgroup sequence. In a still further aspect, the light chain framework sequences are VL kappa I consensus framework. In a still further aspect, one or more of the light chain framework sequences is the following:

(SEQ ID NO: 19)
LC-FR1 DIQMTQSPSSLSASVGDRVTITC
(SEQ ID NO: 20)
LC-FR2 WYQQKPGKAPKLLIY
(SEQ ID NO: 21)
LC-FR3 GVPSRFSGSGSGTDFTLTISSLQPEDFATYYC
(SEQ ID NO: 22)
LC-FR4 FGQGTKVEIKR.

The anti-PD-L1 antibody further can comprise a human or murine constant region. In some embodiments, the human constant region is selected from the group consisting of IgG1, IgG2, IgG2, IgG3, IgG4. In some of these embodiments, the human constant region is IgG1. In other embodiments, the murine constant region is selected from the group consisting of IgG1, IgG2A, IgG2B, IgG3. In some of these embodiments, the murine constant region is IgG2A.

In some embodiments, the anti-PD-L1 antibody has reduced or minimal effector function. In some of these embodiments, the minimal effector function results from an “effector-less Fc mutation” or aglycosylation. In some embodiments, the effector-less Fc mutation is an N297A or D265A/N297A substitution in the constant region.

In other embodiments, the anti-PD-L1 antibody is MPDL3280A (which is described in WO2010/077634, herein incorporated by reference in its entirety) or an antigen-binding fragment thereof. In still other embodiments, the anti-PD-L1 antibody is an antibody that binds to an epitope capable of binding MPDL3280A or competes with MPDL3280A for binding to PD-L1 in a competitive binding assay.

In another further embodiment, the anti-PD-L1 antibody useful in the presently disclosed compositions and methods comprises a heavy chain and a light chain variable region sequence, wherein:

(a) the heavy chain variable region sequence has at least 85% sequence identity to the heavy chain variable region sequence:

(SEQ ID NO: 27)
EVQLVESGGGLVQPGGSLRLSCAASGFTFSDSWIHWVRQAPGKGLEWVAW
ISPYGGSTYYADSVKGRFTISADTSKNTAYLQMNSLRAEDTAVYYCARRH
WPGGFDYWGQGTLVTVSS,

or

(b) the light chain variable region sequence has at least 85% sequence identity to the light chain variable region sequence:

(SEQ ID NO: 26)
DIQMTQSPSSLSASVGDRVTITCRASQDVSTAVAWYQQKPGKAPKLLIY
SASFLYSGVPSRFSGSGSGTDFTLTISSLQPEDFATYYCQQYLYHPATFG
QGTKVEIKR.

In a specific aspect, the sequence identity is 86%, 87%, 88%, 89%, 90%, 91%, 92%, 93%, 94%, 95%, 96%, 97%, 98%, 99% or 100%. In another aspect, the heavy chain variable region comprises one or more framework sequences juxtaposed between the HVRs as: (HC-FR1)-(HVR-H1)-(HC-FR2)-(HVR-H2)-(HC-FR3)-(HVR-H3)-(HC-FR4), and the light chain variable regions comprises one or more framework sequences juxtaposed between the HVRs as: (LC-FR1)-(HVR-L1)-(LC-FR2)-(HVR-L2)-(LC-FR3)-(HVR-L3)-(LC-FR4). In yet another aspect, the framework sequences are derived from human consensus framework sequences. In a further aspect, the heavy chain framework sequences are derived from a Kabat subgroup I, II, or III sequence. In a still further aspect, the heavy chain framework sequence is a VH subgroup III consensus framework. In a still further aspect, one or more of the heavy chain framework sequences is the following:

(SEQ ID NO: 15)
HC-FR1 EVQLVESGGGLVQPGGSLRLSCAAS
(SEQ ID NO: 16)
HC-FR2 WVRQAPGKGLEWV
(SEQ ID NO: 17)
HC-FR3 RFTISADTSKNTAYLQMNSLRAEDTAVYYCAR
(SEQ ID NO: 28)
HC-FR4 WGQGTLVTVSA.

In a still further aspect, the light chain framework sequences are derived from a Kabat kappa I, II, II or IV subgroup sequence. In a still further aspect, the light chain framework sequences are VL kappa I consensus framework. In a still further aspect, one or more of the light chain framework sequences is the following:

(SEQ ID NO: 19)
LC-FR1 DIQMTQSPSSLSASVGDRVTITC
(SEQ ID NO: 20)
LC-FR2 WYQQKPGKAPKLLIY
(SEQ ID NO: 21)
LC-FR3 GVPSRFSGSGSGTDFTLTISSLQPEDFATYYC
(SEQ ID NO: 22)
LC-FR4 FGQGTKVEIKR.

In a still further embodiment, the anti-PD-L1 antibody useful in the presently disclosed compositions and methods comprises a heavy chain comprising the heavy chain amino acid sequence from SEQ ID NO:40 and/or a light chain comprising the light chain amino acid sequence from SEQ ID NO:41. In a still further embodiment, the anti-PD-1 antibody comprises a heavy chain and/or a light chain sequence, wherein:

(a) the heavy chain sequence has at least 85%, at least 90%, at least 91%, at least 92%, at least 93%, at least 94%, at least 95%, at least 96%, at least 97%, at least 98%, at least 99% or 100% sequence identity to the heavy chain sequence:

(SEQ ID NO: 40)
EVQLVESGGGLVQPGGSLRLSCAASGFTFSDSWIHWVRQAPGKGLEWVAW
ISPYGGSTYYADSVKGRFTISADTSKNTAYLQMNSLRAEDTAVYYCARRH
WPGGFDYWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKKVEPKSCDKTHTCPPCPAPELLGGPSVFLFPPKPKD
TLMISRTPEVTCVVVDVSHEDPEVKFNWYVDGVEVHNAKTKPREEQYAST
YRVVSVLTVLHQDWLNGKEYKCKVSNKALPAPIEKTISKAKGQPREPQVY
TLPPSREEMTKNQVSLTCLVKGFYPSDIAVEWESNGQPENNYKTTPPVLD
SDGSFFLYSKLTVDKSRWQQGNVFSCSVMHEALHNHYTQKSLSLSPGK,

or

(b) the light chain sequence has at least 85%, at least 90%, at least 91%, at least 92%, at least 93%, at least 94%, at least 95%, at least 96%, at least 97%, at least 98%, at least 99% or 100% sequence identity to the light chain sequence:

(SEQ ID NO: 41)
DIQMTQSPSSLSASVGDRVTITCRASQDVSTAVAWYQQKPGKAPKLLIYS
ASFLYSGVPSRFSGSGSGTDFTLTISSLQPEDFATYYCQQYLYHPATFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC.

The anti-PD-L1 antibody further can comprise a human or murine constant region. In some embodiments, the human constant region is selected from the group consisting of IgG1, IgG2, IgG2, IgG3, IgG4. In some of these embodiments, the human constant region is IgG1. In other embodiments, the murine constant region is selected from the group consisting of IgG1, IgG2A, IgG2B, IgG3. In some of these embodiments, the murine constant region is IgG2A.

In some embodiments, the anti-PD-L1 antibody has reduced or minimal effector function. In some of these embodiments, the minimal effector function results from an “effector-less Fc mutation” or aglycosylation. In some embodiments, the effector-less Fc mutation is an N297A or D265A/N297A substitution in the constant region.

In other embodiments, the PD-1 axis antagonist useful in the compositions and methods comprises a PD-L2 antagonist. The term “PD-L2 antagonist” refers to a molecule that decreases, blocks, inhibits, abrogates or interferes with signal transduction resulting from the interaction of PD-L2 with either one or more of its binding partners, such as PD-1. In some embodiments, a PD-L2 antagonist is a molecule that inhibits the binding of PD-L2 to its binding partners. In a specific aspect, the PD-L2 antagonist inhibits binding of PD-L2 to PD-1. In some embodiments, the PD-L2 antagonists include anti-PD-L2 antibodies, antigen binding fragments thereof, immunoadhesins, fusion proteins, oligopeptides and other molecules that decrease, block, inhibit, abrogate or interfere with signal transduction resulting from the interaction of PD-L2 with either one or more of its binding partners, such as PD-1. PD-L2 antagonists include molecules that bind to PD-L2 (also referred to herein as PD-L2 binding antagonists) and molecules that reduce the expression of PD-L2, such as the silencing elements described elsewhere herein.

In one embodiment, a PD-L2 antagonist reduces the negative co-stimulatory signal mediated by or through PD-1 cell surface proteins expressed on T lymphocytes so as to enhance effector responses to antigen recognition.

In some embodiments, a PD-L2 antagonist is an immunoadhesin, such as a polypeptide that comprises the extracellular or PD-L2 binding portions of PD-1, fused to a constant domain of an immunoglobulin sequence (e.g., Fc).

In some embodiments, the anti-PD-L2 antibody is a monoclonal antibody. In some embodiments, the anti-PD-L2 antibody is an antibody fragment selected from the group consisting of Fab, Fab′-SH, Fv, scFv, and (Fab′)₂ fragments. In some embodiments, the anti-PD-L2 antibody is a humanized antibody. In some embodiments, the anti-PD-L2 antibody is a human antibody.

In a still further specific aspect, an antibody described herein (such as an anti-PD-1 antibody, an anti-PD-L1 antibody, or an anti-PD-L2 antibody) further comprises a human or murine constant region. In a still further aspect, the human constant region is selected from the group consisting of IgG1, IgG2, IgG2, IgG3, IgG4. In a still further specific aspect, the human constant region is IgG1. In a still further aspect, the murine constant region is selected from the group consisting of IgG1, IgG2A, IgG2B, IgG3. In a still further aspect, the murine constant region if IgG2A. In a still further specific aspect, the antibody has reduced or minimal effector function. In a still further specific aspect, the minimal effector function results from production in prokaryotic cells. In a still further specific aspect the minimal effector function results from an “effector-less Fc mutation” or aglycosylation. In still a further aspect, the effector-less Fc mutation is an N297A or D265A/N297A substitution in the constant region.

In a still further aspect, provided herein are nucleic acids encoding any of the anti-PD-1, anti-PD-L1, or anti-PD-L2 antibodies described herein. In some embodiments, the nucleic acid further comprises a vector suitable for expression of the nucleic acid encoding any of the described anti-PD-L1, anti-PD-1, or anti-PD-L2. In a still further specific aspect, the vector further comprises a host cell suitable for expression of the nucleic acid. In a still further specific aspect, the host cell is a eukaryotic cell or a prokaryotic cell. In a still further specific aspect, the eukaryotic cell is a mammalian cell, such as Chinese Hamster Ovary (CHO).

The antibody or antigen binding fragment thereof, may be made using methods known in the art, for example, by a process comprising culturing a host cell containing nucleic acid encoding any of the previously described anti-PD-L1, anti-PD-1, or anti-PD-L2 antibodies in a form suitable for expression, under conditions suitable to produce such an antibody, and recovering the antibody.

In some embodiments, the PD-1 antagonist, PD-L1 antagonist, or PD-L2 antagonist comprises an oligopeptide. A “PD-1 oligopeptide,” “PD-L1 oligopeptide,” or “PD-L2 oligopeptide” is an oligopeptide that binds, in some embodiments, specifically, to a PD-1, PD-L1 or PD-L2 negative costimulatory polypeptide, respectively, including a receptor, ligand or signaling component, respectively, as described herein. Such oligopeptides may be chemically synthesized using known oligopeptide synthesis methodology or may be prepared and purified using recombinant technology. Such oligopeptides are usually at least about 5 amino acids in length, alternatively at least about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acids in length or more. Such oligopeptides may be identified using well known techniques. In this regard, it is noted that techniques for screening oligopeptide libraries for oligopeptides that are capable of specifically binding to a polypeptide target are well known in the art (see, e.g., U.S. Pat. Nos. 5,556,762, 5,750,373, 4,708,871, 4,833,092, 5,223,409, 5,403,484, 5,571,689, 5,663,143; PCT Publication Nos. WO 84/03506 and WO84/03564; Geysen et al., Proc. Natl. Acad. Sci. U.S.A., 81:3998-4002 (1984); Geysen et al., Proc. Natl. Acad. Sci. U.S.A., 82:178-182 (1985); Geysen et al., in Synthetic Peptides as Antigens, 130-149 (1986); Geysen et al., J. Immunol. Meth., 102:259-274 (1987); Schoofs et al., J. Immunol., 140:611-616 (1988), Cwirla, S. E. et al. Proc. Natl. Acad. Sci. USA, 87:6378 (1990); Lowman, H. B. et al. Biochemistry, 30:10832 (1991); Clackson, T. et al. Nature, 352: 624 (1991); Marks, J. D. et al., J. Mol. Biol., 222:581 (1991); Kang, A. S. et al. Proc. Natl. Acad. Sci. USA, 88:8363 (1991), and Smith, G. P., Current Opin. Biotechnol., 2:668 (1991).

B. HPK1 Antagonists

The hematoipoietic progenitor kinase 1 or HPK1, also referred to as mitogen activated protein kinase kinase kinase kinase 1 or MAP4K1, is a member of the germinal center kinase subfamily of Ste20-related serine/threnonine kinases. HPK1 polynucleotides and polypeptides are known in the art (Hu et al. (1996) Genes Dev. 10: 2251-2264, which is herein incorporated by reference in its entirety). Non-limiting examples of HPK1 polynucleotides and polypeptides comprise the human HPK1 polynucleotide as set forth in SEQ ID NO: 11 (nucleotides 141-2642 of GenBank Accession No. NM_007181.5) and the encoded human HPK1 polypeptide (Accession No. NP_009112.1) as set forth in SEQ ID NO: 12. A shorter 821 amino acid isoform of HPK1 exists in humans, the coding sequence and amino acid sequence of which is set forth in SEQ ID NOs: 13 and 14, respectively (nucleotides 141-2606 of GenBank Accession No. NM_001042600.2 and GenBank Accession No. NP_001036065.1, respectively).

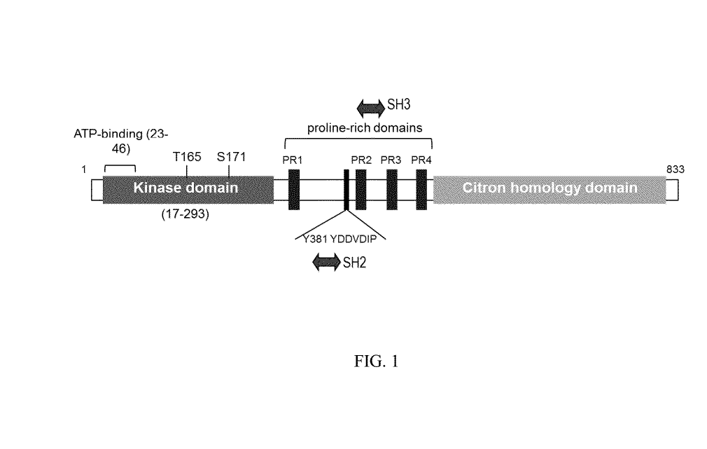

HPK1 polypeptides comprise a variety of conserved structural motifs. For ease of reference, such motifs will be discussed as they relate to the longer human HPK1 isoform, which is set forth in SEQ ID NO:12, comprises 833 amino acid residues, and is depicted in FIG. 1. HPK1 polypeptides comprise an amino-terminal Ste20-like kinase domain that spans amino acid residues 17-293, which includes the ATP-binding site from amino acid residues 23-46. The kinase domain is followed by four proline-rich (PR) motifs that serve as binding sites for SH3-containing proteins, such as CrkL, Grb2, HIP-55, Gads, Nck, and Crk. The four PR motifs span amino acid residues 308-407, 394-402, 432-443, and 468-477, respectively. HPK1 becomes phosphorylated and activated in response to TCR or BCR stimulation. TCR- and BCR-induced phosphorylation of the tyrosine at position 381, located between PR1 and PR2, mediates binding to SLP-76 in T cells or BLNK in B cells via a SLP-76 or BLNK SH2 domain, and is required for activation of the kinase. A citron homology domain found in the C-terminus of HPK1, approximately spanning residues 495-800, may act as a regulatory domain and may be involved in macromolecular interactions.

Although HPK1 is expressed in all embryonic tissues, postnatally, its expression is primarily restricted to hematopoietic organs and cells. HPK1 functions as a MAP4K by phosphorylating and activating MAP3K proteins, including MEKK1, MLK3 and TAK1, leading to the activation of the MAPK Jnk.

HPK1 is a negative regulator of T and B cell responses. In T cells, it is believed that HPK1 negatively regulates T cell activation by reducing the persistence of signaling microclusters by phosphorylating SLP76 at Ser376 (Di Bartolo et al. (2007) JEM 204:681-691) and Gads at Thr254, which leads to the recruitment of 14-3-3 proteins that bind to the phosphorylated SLP76 and Gads, releasing the SLP76-Gads-14-3-3 complex from LAT-containing microclusters (Lasserre et al. (2011) J Cell Biol 195(5):839-853). HPK1 can also become activated in response to prostaglandin E2, which is often secreted by tumors, contributing to the escape of tumor cells from the immune system.

The presently disclosed compositions comprise both a PD-1 axis antagonist and a HPK1 antagonist that can be used to enhance an immune response and to treat cancer by inhibiting tumor growth.

As used herein, a “HPK1 antagonist” is a molecule that reduces, inhibits, or otherwise diminishes one or more of the biological activities of HPK1 (e.g., serine/threonine kinase activity, recruitment to the TCR complex upon TCR activation, interaction with a protein binding partner, such as SLP76). Antagonism using the HPK1 antagonist does not necessarily indicate a total elimination of the HPK1 activity. Instead, the activity could decrease by a statistically significant amount including, for example, a decrease of at least about 5%, 10%, 15%, 20%, 25%, 30%, 35%, 40%, 45%, 50%, 55%, 60%, 65%, 70%, 75%, 80%, 85%, 95% or 100% of the activity of HPK1 compared to an appropriate control. In some embodiments, the HPK1 antagonist reduces, inhibits, or otherwise diminishes the serine/threonine kinase activity of HPK1. In some of these embodiments, the HPK1 antagonist reduces, inhibits, or otherwise diminishes the HPK1-mediated phosphorylation of SLP76 and/or Gads.

As shown herein, any inhibition of HPK1 when combined with a PD1 axis antagonist provides excellent anti-tumor efficacy. Useful HPK1 antagonists, which also can be identified as mitogen-activated protein kinase kinase kinase kinase 1 antagonists or MAP4K1 antagonists, include those that show inhibition as described above and as determined by any assay methods in the art. Specific HPK1 inhibitors include those that exhibit inhibitory activity in the MC38 model described elsewhere herein. In particularly useful embodiments, the HPK1 inhibitor is a small molecule inhibitor as described elsewhere herein. Many such inhibitors are known compounds. Routine screening of known compounds can identify compounds that are inhibitors of HPK1. Compounds include heteroaryl compounds that bind to the hinge region of the enzyme. A HPK1 antagonist may bind, directly or indirectly, to HPK1, inhibiting its activity, or an HPK1 antagonist may function to reduce or inhibit the expression of HPK1, such as a HPK1 silencing element, which is described in more detail elsewhere herein.

For example, HPK1 antagonists include anti-HPK1 intrabodies and other molecules that decrease, block, inhibit, abrogate or interfere with a biological activity of HPK1.

The HPK1 antagonist can be a small molecule, which can be an organic or inorganic compound (i.e., including heteroorganic and organometallic compounds). The HPK1 antagonist can also be a peptide, peptidomimetic, amino acid, amino acid analog, polynucleotide, polynucleotide analog, nucleotide, nucleotide analog, or a lipid. In some embodiments, small molecules have a weight of less than about 10,000, 5,000, 1,000, or 500 grams per mole.

Further, the HPK1 antagonist may or may not be a specific HPK1 antagonist. A specific HPK1 antagonist reduces the biological activity of HPK1 by an amount that is statistically greater than the inhibitory effect of the antagonist on any other protein (e.g., other serine/threonine kinases). In certain embodiments, the HPK1 antagonist specifically inhibits the serine/threonine kinase activity of HPK1. In some of these embodiments, the IC₅₀ of the HPK1 antagonist for HPK1 is about 90%, 80%, 70%, 60%, 50%, 40%, 30%, 20%, 10%, 0.1%, 0.01%, 0.001%, or less of the IC₅₀ of the HPK1 antagonist for another serine/threonine kinase or other type of kinase (e.g., tyrosine kinase).

An antagonist of HPK1 that inhibits the serine-threonine kinase activity of HPK1 may be a competitive inhibitor, preventing the binding of the substrate (ATP or protein substrate), non-competitive inhibitors, binding to the enzyme whether or not substrate (ATP or protein substrate) is also bound, or un-competitive inhibitors that only bind to the enzyme once bound to substrate (ATP and protein substrate). The HPK1 antagonist may be an allosteric inhibitor, binding to a site on HPK1 other than the active site.

Antagonists may function as a competitive inhibitor by binding within the substrate-binding domain (ATP-binding domain or protein substrate-binding domain), thus blocking binding of the substrate (ATP or protein substrate). Alternatively, competitive inhibitors can function as allosteric inhibitors and bind to sites outside of the substrate binding site of the free enzyme, blocking binding of the substrate (ATP or protein substrate).

In some embodiments, the HPK1 antagonist is a competitive inhibitor of HPK1. In some of these embodiments, the HPK1 antagonist is a competitive inhibitor that binds to the ATP binding site of HPK1 when HPK1 is in an active conformation, inhibiting binding of ATP and functioning as an ATP mimic. In other embodiments, the HPK1 antagonist binds to an inactive conformation of HPK1.

HPK1 small molecule antagonists are known in the art and include, but are not limited to, staurosporine, bosutinib, sunitinib, lestaurtinib, crizotinib, foretinib, dovitinib, and KW-2449 (Davis et al. (2011) Nat Biotechnol 29(11):1046-1051; Wodicka et al. (2010) Chem Biol 17(11):1241-1249, each of which is herein incorporated by reference in its entirety).

Due to the cytoplasmic location of HPK1, antibodies are not effective HPK1 antagonists. However, in some embodiments, the HPK1 antagonist can be a polynucleotide that encodes an intrabody. When the polynucleotide is introduced into and expressed within a cell expressing HPK1, the intrabody binds to HPK1 and antagonizes its activity. The intrabody binds to HPK1 and inhibits a biological activity, such as kinase activity, recruitment to the TCR complex upon TCR activation, or interaction with a protein binding partner, such as SLP76.

In other embodiments, the HPK1 antagonist is a polynucleotide that encodes a peptide that is capable of binding to HPK1 and inhibiting its activity. When such a polynucleotide is introduced into and expressed within a cell that expresses HPK1, the intracellular peptide binds to and inhibits HPK1 activity.

In yet other embodiments, the HPK1 antagonist is a polynucleotide that mediates site-directed mutagenesis through homologous recombination of an HPK1 gene to generate a kinase dead HPK1 protein. For example, as demonstrated herein, mutation of the conserved lysine at amino acid position 46 in both human and murine HPK1 to glutamate produces a kinase dead HPK1 protein.

The HPK1 and/or PD-1 axis antagonist may comprise a silencing element. As used herein, the term “silencing element” refers to a polynucleotide, which when expressed or introduced into a cell is capable of reducing or eliminating the level of expression of a target polynucleotide sequence or the polypeptide encoded thereby. In some embodiments, the silencing element can be operably linked to a promoter to allow expression of the silencing element in a cell.

In one embodiment, the silencing element encodes a zinc finger protein that binds to a HPK1, PD-1, PD-L1, or PD-L2 gene, resulting in reduced expression of the gene. In particular embodiments, the zinc finger protein binds to a regulatory region of a HPK1, PD-1, PD-L1, or PD-L2 gene. In other embodiments, the zinc finger protein binds to a messenger RNA encoding a HPK1, PD-1, PD-L1, or PD-L2 and prevents its translation. Methods of selecting sites for targeting by zinc finger proteins have been described, for example, in U.S. Pat. No. 6,453,242, which is herein incorporated by reference.

In some embodiments, the activity of HPK1, PD-1, PD-L1, or PD-L2 is reduced or eliminated by disrupting a HPK1, PD-1, PD-L1, or PD-L2 gene, respectively. The HPK1, PD-1, PD-L1, or PD-L2 gene may be disrupted by any method known in the art. For example, in one embodiment, the gene is disrupted by transposon tagging. In another embodiment, the gene is disrupted by mutagenizing cells using random or targeted mutagenesis, and selecting for cells that have reduced HPK1, PD-1, PD-L1, or PD-L2 activity.

In one embodiment, transposon tagging is used to reduce or eliminate the activity of HPK1, PD-1, PD-L1, or PD-L2. Transposon tagging comprises inserting a transposon within an endogenous HPK1, PD-1, PD-L1, or PD-L2 gene to reduce or eliminate expression of the HPK1, PD-1, PD-L1, or PD-L2. In this embodiment, the expression of the HPK1, PD-1, PD-L1, or PD-L2 gene is reduced or eliminated by inserting a transposon within a regulatory region or coding region of the HPK1, PD-1, PD-L1, or PD-L2 gene. A transposon that is within an exon, intron, 5′ or 3′ untranslated sequence, a promoter, or any other regulatory sequence of a HPK1, PD-1, PD-L1, or PD-L2 gene may be used to reduce or eliminate the expression and/or activity of the encoded HPK1, PD-1, PD-L1, or PD-L2, respectively. In these embodiments, the silencing element comprises or encodes a targeted transposon that can insert within a HPK1, PD-1, PD-L1, or PD-L2 gene.

In other embodiments, the silencing element comprises a nucleotide sequence useful for site-directed mutagenesis via homologous recombination within a region of a HPK1, PD-1, PD-L1, or PD-L2 gene. Insertional mutations in gene exons usually result in null mutants. Additional methods for reducing or eliminating the activity or expression of HPK1, PD-1, PD-L1, or PD-L2 may be used, such as those that involve promoter-based silencing. See, for example, Mette et al. (2000) EMBO J. 19: 5194-5201; Sijen et al. (2001) Curr. Biol. 11: 436-440; Jones et al. (2001) Curr. Biol. 11: 747-757, each of which are herein incorporated by reference in its entirety.

The silencing element can comprise or encode an antisense oligonucleotide or an interfering RNA (RNAi). The term “interfering RNA” or “RNAi” refers to any RNA molecule which can enter an RNAi pathway and thereby reduce the expression of a target gene, such as small RNA (sRNA), short-interfering RNA (siRNA), micro-RNA (miRNA), double-stranded RNA (d5RNA), hairpin RNA, short hairpin RNA (shRNA), and others. See, for example, Meister and Tuschl (2004) Nature 431:343-349; Bonetta et al. (2004) Nature Methods 1:79-86; Chuang and Meyerowitz (2000) Proc. Natl. Acad. Sci. USA 97:4985-4990; McManus et al. (2002) Nature Reviews Genetics 3: 737-747; Dykxhoorn et al. (2003) Nature Reviews Molecular Cell Biology 4: 457-467. A variety of computer programs also are available to assist with selection of siRNA sequences, e.g., from Ambion (web site having URL www.ambion.com), at the web site having the URL www.sinc.sunysb.edu/Stu/shilin/rnai.html. Additional design considerations that also can be employed are described in Semizarov et al. Proc. Natl. Acad. Sci. 100: 6347-6352.

In some embodiments, the silencing element comprises or encodes an antisense oligonucleotide. An “antisense oligonucleotide” is a single-stranded nucleic acid sequence that is wholly or partially complementary to a target gene, and can be DNA, or its RNA counterpart (i.e., wherein T residues of the DNA are U residues in the RNA counterpart).

The antisense oligonucleotides useful in the presently disclosed compositions and methods are designed to be hybridizable with target RNA (e.g., mRNA) or DNA. For example, an oligonucleotide (e.g., DNA oligonucleotide) that hybridizes to an mRNA molecule can be used to target the mRNA for RnaseH digestion. Alternatively, an oligonucleotide that hybridizes to the translation initiation site of an mRNA molecule can be used to prevent translation of the mRNA. In another approach, oligonucleotides that bind to double-stranded DNA can be administered. Such oligonucleotides can form a triplex construct and inhibit the transcription of the DNA. Triple helix pairing prevents the double helix from opening sufficiently to allow the binding of polymerases, transcription factors, or regulatory molecules. Such oligonucleotides can be constructed using the base-pairing rules of triple helix formation and the nucleotide sequences of the target genes.

As non-limiting examples, antisense oligonucleotides can be targeted to hybridize to the following regions: mRNA cap region, translation initiation site, translational termination site, transcription initiation site, transcription termination site, polyadenylation signal, 3′ untranslated region, 5′ untranslated region, 5′ coding region, mid coding region, and 3′ coding region. In some embodiments, the complementary oligonucleotide is designed to hybridize to the most unique 5′ sequence of a gene, including any of about 15-35 nucleotides spanning the 5′ coding sequence. Antisense nucleic acids can be produced by standard techniques (see, for example, Shewmaker et al., U.S. Pat. No. 5,107,065). Appropriate oligonucleotides can be designed using OLIGO software (Molecular Biology Insights, Inc., Cascade, Colo.; http://www.oligo.net).

The silencing elements employed in the presently disclosed methods and compositions can comprise a DNA template for a dsRNA (e.g., shRNA) or antisense RNA. In such embodiments, the DNA molecule encoding the dsRNA or antisense RNA is found in an expression cassette. In addition, polynucleotides that comprise a coding sequence for a polypeptide or antibody (e.g., antibody that inhibits HPK1, PD-1, PD-L1, or PD-L2 activity) can be found in an expression cassette.

The expression cassettes can comprise one or more regulatory sequences that are operably linked to the nucleotide sequence encoding the silencing element, polypeptide, or antibody that facilitate expression of the polynucleotide or polypeptide. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. See, for example, Goeddel (1990) in Gene Expression Technology: Methods in Enzymology 185 (Academic Press, San Diego, Calif.). Regulatory sequences may include promoters, translation leader sequences, introns, and polyadenylation recognition sequences.

Regulatory sequences are operably linked with a coding sequence to allow for expression of the polypeptide encoded by the coding sequence or to allow for the expression of the encoded polynucleotide silencing element. “Operably linked” is intended to mean that the coding sequence (i.e., a DNA encoding a silencing element or a coding sequence for a polypeptide of interest) is functionally linked to the regulatory sequence(s) in a manner that allows for expression of the nucleotide sequence. Operably linked elements may be contiguous or non-contiguous. Polynucleotides may be operably linked to regulatory sequences in sense or antisense orientation.

The regulatory regions (i.e., promoters, transcriptional regulatory regions, and translational termination regions) and/or the coding polynucleotides may be native/analogous to the cell to which the polynucleotide is being introduced or to each other. Alternatively, the regulatory regions and/or the coding polynucleotides may be heterologous to the cell to which the polynucleotide is being introduced or to each other.

As used herein, “heterologous” in reference to a sequence is a sequence that originates from a foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide. Alternatively, a sequence that is heterologous to a cell is a sequence that originates from a foreign species, or, if from the same species, is substantially modified in the cell from its native form in composition and/or genomic locus by deliberate human intervention.

In general, expression cassettes of utility in recombinant DNA techniques are often in the form of plasmids (vectors). However, other forms of expression cassettes, such as viral vectors (e.g., replication defective retroviruses, adenoviruses, lentiviruses, and adeno-associated viruses) may be used. See, for example, U.S. Publication 2005214851, herein incorporated by reference. Retroviral vectors, particularly lentiviral vectors, are transduced by packaging the vectors into virions prior to contact with a cell.

An expression cassette can further comprise a selection marker. As used herein, the term “selection marker” comprises any polynucleotide, which when expressed in a cell allows for the selection of the transformed cell with the vector.

Such methods involve introducing a polypeptide or polynucleotide into a cell.

“Introducing” is intended to mean presenting to the cell the polynucleotide or polypeptide in such a manner that the sequence gains access to the interior of a cell. The presently disclosed methods do not depend on a particular method for introducing a sequence into a cell, only that the polynucleotide or polypeptides gains access to the interior of a cell. Methods for introducing polynucleotide or polypeptides into various cell types are known in the art including, but not limited to, stable transformation methods, transient transformation methods, and virus-mediated methods.

Exemplary art-recognized techniques for introducing foreign polynucleotides into a host cell, include, but are not limited to, calcium phosphate or calcium chloride co-precipitation, DEAE-dextran-mediated transfection, lipofection, particle gun, or electroporation and viral vectors. Suitable methods for transforming or transfecting host cells can be found in U.S. Pat. No. 5,049,386, U.S. Pat. No. 4,946,787; and U.S. Pat. No. 4,897,355, Sambrook et al. (1989) Molecular Cloning: A Laboratory Manual (2d ed., Cold Spring Harbor Laboratory Press, Plainview, N.Y.) and other standard molecular biology laboratory manuals. Various transfection agents can be used in these techniques. Such agents are known, see for example, WO 2005012487. One of skill will recognize that depending on the method by which a polynucleotide is introduced into a cell, the silencing element can be stably incorporated into the genome of the cell, replicated on an autonomous vector or plasmid, or presented transiently in the cell. Viral vector delivery systems include DNA and RNA viruses, which have either episomal or integrated genomes after delivery to the cell. For a review of viral vector procedures, see Anderson (1992) Science 256:808-813; Haddada et al. (1995) Current Topics in Microbiology and Immunology Doerfler and Bohm (eds); and Yu et al. (1994) Gene Therapy 1:13-26. Conventional viral based systems for the delivery of polynucleotides could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene.

The presently disclosed compositions and methods comprising a PD-1 axis antagonist and a HPK-1 antagonist, wherein at least one of the antagonists is a polynucleotide can utilize in vivo gene therapy, wherein the polynucleotide is introduced into a cell within a subject via administration of the polynucleotide to the subject or ex vivo gene therapy, wherein the polynucleotide is introduced into a cell outside of a subject and then the cell comprising the polynucleotide is administered to a subject. In some of the embodiments wherein at least one of the PD-1 axis antagonist and HPK-1 antagonist is introduced into a cell ex vivo, the cell in which the polynucleotide is introduced and is subsequently administered to a subject is an autologous, allogeneic, or xenogeneic cell with respect to the subject. In some of those embodiments wherein ex vivo gene therapy is utilized, the cell in which the polynucleotide is introduced is a stem cell, such as a hematopoietic stem cell, or a hematopoietic progenitor cell. In other embodiments, the cell in which the polynucleotide is introduced ex vivo is a T cell, B cell, or dendritic cell.

C. Pharmaceutical Compositions

The PD-1 axis antagonist and/or HPK1 antagonist may be in a pharmaceutical composition or formulation. In some embodiments, the pharmaceutical composition or formulation comprises one or more HPK1 antagonists and/or one or more of the PD-1 axis antagonists described herein and a pharmaceutically acceptable carrier.

The phrase “pharmaceutically acceptable” indicates that the substance or composition must be compatible chemically and/or toxicologically, with the other ingredients comprising a formulation, and/or the subject being treated therewith.

“Carriers” as used herein include pharmaceutically acceptable carriers, excipients, or stabilizers that are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™. In certain embodiments, the pharmaceutical composition comprises a non-naturally occurring pharmaceutically acceptable carrier.

A pharmaceutical composition is formulated to be compatible with its intended route of administration. In some embodiments, the active compound(s) are delivered in a vesicle, such as liposomes (see, e.g., Langer (1990) Science 249:1527-33; and Treat et al., in Liposomes in the Therapy of Infectious Disease and Cancer, Lopez Berestein and Fidler (eds.), Liss, N.Y., pp. 353-65, 1989).

In yet another embodiment, the active compound(s) can be delivered in a controlled release system. In one example, a pump can be used (see, e.g., Langer (1990) Science 249:1527-33; Sefton (1987) Crit. Rev. Biomed. Eng. 14:201-40; Buchwald et al. (1980) Surgery 88:507-16; Saudek et al. (1989) N. Engl. J. Med. 321:574-79). In another example, polymeric materials can be used (see, e.g., Levy et al. (1985) Science 228:190-92; During et al. (1989) Ann. Neurol. 25:351-56; Howard et al. (1989) J. Neurosurg. 71:105-12). Other controlled release systems, such as those discussed by Langer (1990) Science 249:1527-33, can also be used.

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes, or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor® EL (BASF; Parsippany, N.J.), or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion, and by the use of surfactants. Prevention of the action of microorganisms can be achieved by various antibacterial and antifungal agents, for example, parabens, chlorobutanol, phenol, ascorbic acid, thimerosal, and the like. In many cases, isotonic agents are included, for example, sugars, polyalcohols such as mannitol, sorbitol, sodium chloride, in the composition. Prolonged absorption of the injectable compositions can be brought about by including in the composition an agent that delays absorption, for example, aluminum monostearate and gelatin.

Sterile injectable solutions can be prepared by incorporating the active compound(s) in the required amount in an appropriate solvent with one or a combination of ingredients enumerated above, as required, followed by filtered sterilization. Generally, dispersions are prepared by incorporating the active compound(s) into a sterile vehicle that contains a basic dispersion medium and the required other ingredients from those enumerated above. In the case of sterile powders for the preparation of sterile injectable solutions, the methods of preparation can include vacuum drying and freeze-drying, which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound(s) can be incorporated with excipients and used in the form of tablets, troches, or capsules. Oral compositions can also be prepared using a fluid carrier for use as a mouthwash, wherein the compound(s) in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth, or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. For administration by inhalation, the compound(s) are delivered in the form of an aerosol spray from a pressurized container or dispenser that contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compound(s) are formulated into ointments, salves, gels, or creams as generally known in the art. The compound(s) can also be prepared in the form of suppositories (e.g., with conventional suppository bases such as cocoa butter and other glycerides) or retention enemas for rectal delivery.

In one embodiment, the active compound(s) are prepared with carriers that will protect the compound(s) against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated with each unit containing a predetermined quantity of active compound calculated to produce the desired therapeutic effect in association with the required pharmaceutical carrier. The specification for the dosage unit forms of the compounds are dictated by and directly dependent on the unique characteristics of the active compound and the particular therapeutic effect to be achieved, and the limitations inherent in the art of compounding such an active compound for the treatment of subjects.

D. Kits

In another aspect, provided is a kit comprising a PD-1 axis antagonist and/or a HPK1 antagonist, and a package insert with instructions for use. A “package insert” refers to instructions customarily included in commercial packages of medicaments that contain information about the indications customarily included in commercial packages of medicaments that contain information about the indications, usage, dosage, administration, contraindications, other medicaments to be combined with the packaged product, and/or warnings concerning the use of such medicaments, etc.

In some embodiments, the kit comprises a PD-1 axis antagonist and a package insert comprising instructions for using the PD-1 axis antagonist in combination with a HPK1 antagonist to enhance an immune response or to treat cancer in a subject in need thereof. In some embodiments, the kit comprises a HPK1 antagonist and a package insert comprising instructions for using the HPK1 antagonist in combination with a PD-1 axis antagonist to enhance an immune response or to treat cancer in a subject in need thereof. In some embodiments, the kit comprises a PD-1 axis antagonist and a HPK1 antagonist, and a package insert comprising instructions for using the PD-1 axis antagonist and the HPK1 antagonist to enhance an immune response or to treat cancer in a subject in need thereof. Any of the PD-1 axis antagonists and/or HPK1 antagonists described herein may be included in the kits.

In some embodiments, the kit comprises a container containing one or more of the PD-1 axis antagonists and HPK1 antagonists described herein. Suitable containers include, for example, bottles, vials (e.g., dual chamber vials), syringes (such as single or dual chamber syringes) and test tubes. The container may be formed from a variety of materials such as glass or plastic. In some embodiments, the kit may comprise a label (e.g., on or associated with the container). The label may indicate that the compound contained therein may be useful or intended for enhancing an immune response or treating cancer in a subject in need thereof. The kit may further comprise other materials desirable from a commercial and user standpoint, including other buffers, diluents, filters, needles, and syringes.

In some embodiments, the kit further comprises a chemotherapeutic agent, including but not limited to those described elsewhere herein.

E. Methods of Using PD-1 Axis Antagonists and HPK-1 Antagonists

Provided herein is a method of enhancing an immune response in a subject in need thereof comprising administering an effective amount of a PD-1 axis antagonist and a HPK1 antagonist.

As used herein, “enhancing an immune response” refers to an improvement in any immunogenic response to an antigen. Non-limiting examples of improvements in an immunogenic response to an antigen include enhanced maturation or migration of dendritic cells, enhanced activation of T cells (e.g., CD4 T cells, CD8 T cells), enhanced T cell (e.g., CD4 T cell, CD8 T cell) proliferation, enhanced B cell proliferation, increased survival of T cells and/or B cells, improved antigen presentation by antigen presenting cells (e.g., dendritic cells), improved antigen clearance, increase in production of cytokines by T cells (e.g., interleukin-2), increased resistance to prostaglandin E2-induced immune suppression, and enhanced priming and/or cytolytic activity of CD8 T cells.

In some embodiments, the CD8 T cells in the subject have enhanced priming, activation, proliferation and/or cytolytic activity relative to prior to the administration of the PD-1 pathway antagonist and the HPK1 antagonist. In some embodiments, the CD8 T cell priming is characterized by elevated CD44 expression and/or enhanced cytolytic activity in CD8 T cells. In some embodiments, the CD8 T cell activation is characterized by an elevated frequency of γ-IFN⁺ CD8 T cells. In some embodiments, the CD8 T cell is an antigen-specific T-cell. In some embodiments, the immune evasion by signaling through PD-L1 surface expression is modulated.

In some embodiments, the antigen presenting cells in the subject have enhanced maturation and activation relative to prior to the administration of the PD-1 pathway antagonist and the HPK1 antagonist. In some embodiments, the antigen presenting cells are dendritic cells. In some embodiments, the maturation of the antigen presenting cells is characterized by an increased frequency of CD83⁺ dendritic cells. In some embodiments, the activation of the antigen presenting cells is characterized by elevated expression of CD80 and CD86 on dendritic cells.

In some embodiments, the serum levels of cytokine IL-10 and/or chemokine IL-8, a human homolog of murine KC, in the subject are reduced relative to prior to the administration of the PD-1 antagonist and the HPK1 antagonist.

PD-L1 or PD-L2 binding to PD-1 results in the tyrosine phosphorylation of the PD-1 cytoplasmic domain and subsequent recruitment of phosphatases, including SHP2, which results in the dephosphorylation of ZAP70 and other TCR proximal signaling molecules, leading to attenuation of TCR signaling and T cell dysfunction, including anergy and exhaustion (Chemnitz et al. (2004) J Immunol 173(2):945-954).

The term “dysfunction” in the context of immune dysfunction, refers to a state of reduced immune responsiveness to antigenic stimulation. The term includes the common elements of both exhaustion and/or anergy in which antigen recognition may occur, but the ensuing immune response is ineffective to control infection or tumor growth.

The term “dysfunctional”, as used herein, also includes refractory or unresponsive to antigen recognition, specifically, impaired capacity to translate antigen recognition into down-stream T-cell effector functions, such as proliferation, cytokine production (e.g., IL-2, γ-IFN) and/or target cell killing.

The term “anergy” refers to the state of unresponsiveness to antigen stimulation resulting from incomplete or insufficient signals delivered through the T-cell receptor (e.g. increase in intracellular Ca⁺² in the absence of ras-activation). T cell anergy can also result upon stimulation with antigen in the absence of co-stimulation, resulting in the cell becoming refractory to subsequent activation by the antigen even in the context of costimulation. The unresponsive state can often be overriden by the presence of Interleukin-2 Anergic T-cells do not undergo clonal expansion and/or acquire effector functions.

The term “exhaustion” refers to T cell exhaustion as a state of T cell dysfunction that arises from sustained TCR signaling that occurs during many chronic infections and cancer. It is distinguished from anergy in that it arises not through incomplete or deficient signaling, but from sustained signaling. It is defined by poor effector function, sustained expression of inhibitory receptors and a transcriptional state distinct from that of functional effector or memory T cells. Exhaustion prevents optimal control of infection and tumors. Exhaustion can result from both extrinsic negative regulatory pathways (e.g., immunoregulatory cytokines) as well as cell intrinsic negative regulatory (costimulatory) pathways (PD-1, B7-H3, B7-H4, etc.).

In some embodiments, administration of a PD-1 axis antagonist and HPK1 antagonist to a subject results in an enhancement of T cell function.

“Enhancing T cell function” means to induce, cause or stimulate a T cell to have a sustained or amplified biological function, or renew or reactivate exhausted or inactive T cells. Examples of enhancing T cell function include: increased secretion of cytokines (e.g., γ-interferon, IL-2, IL-12, and TNFα), increased proliferation, increased antigen responsiveness (e.g., viral, pathogen, or tumor clearance) relative to such levels before the intervention, and increased effector granule production by CD8 T cells, such as granzyme B. In one embodiment, the level of enhancement is as least 50%, alternatively 60%, 70%, 80%, 90%, 100%, 120%, 150%, 200%. The manner of measuring this enhancement is known to one of ordinary skill in the art.

Accordingly, the combination therapy of a PD-1 axis antagonist and a HPK1 antagonist are useful in treating T cell dysfunctional disorders. A “T cell dysfunctional disorder” is a disorder or condition of T cells characterized by decreased responsiveness to antigenic stimulation. In a particular embodiment, a T cell dysfunctional disorder is a disorder that is specifically associated with inappropriate increased signaling through PD-1 and/or inappropriate increased kinase activity of HPK1. In another embodiment, a T cell dysfunctional disorder is one in which T cells are anergic or have decreased ability to secrete cytokines, proliferate, or execute cytolytic activity. In a specific aspect, the decreased responsiveness results in ineffective control of a pathogen or tumor expressing an immunogen. Examples of T cell dysfunctional disorders characterized by T-cell dysfunction include unresolved acute infection, chronic infection and tumor immunity.

Thus, the presently disclosed combination therapy of a PD-1 axis antagonist and a HPK1 antagonist can be used in treating conditions where enhanced immunogenicity is desired, such as increasing tumor immunogenicity for the treatment of cancer.

“Immunogenecity” refers to the ability of a particular substance to provoke an immune response. Tumors are immunogenic and enhancing tumor immunogenicity aids in the clearance of the tumor cells by the immune response.

“Tumor immunity” refers to the process in which tumors evade immune recognition and clearance. Thus, as a therapeutic concept, tumor immunity is “treated” when such evasion is attenuated, and the tumors are recognized and attacked by the immune system. Examples of tumor recognition include tumor binding, tumor shrinkage and tumor clearance.

In one aspect, provided herein is a method for treating of cancer in a subject in need thereof comprising administering to the subject an effective amount of a PD-1 axis antagonist and a HPK1 antagonist.

The term “cancer” and “cancerous” refer to the condition in a subject that is characterized by unregulated cell growth, wherein the cancerous cells are capable of local invasion and/or metastasis to noncontiguous sites. Included in this definition are benign and malignant cancers. As used herein, “cancer cells,” “cancerous cells,” or “tumor cells” refer to the cells that are characterized by this unregulated cell growth and invasive property. The term “cancer” encompasses all types of cancers, including, but not limited to, all forms of carcinomas, melanomas, sarcomas, lymphomas and leukemias, including without limitation, bladder carcinoma, brain tumors, breast cancer, cervical cancer, colorectal cancer, esophageal cancer, endometrial cancer, hepatocellular carcinoma, laryngeal cancer, lung cancer, osteosarcoma, ovarian cancer, pancreatic cancer, prostate cancer, renal carcinoma and thyroid cancer, acute lymphocytic leukemia, acute myeloid leukemia, ependymoma, Ewing's sarcoma, glioblastoma, medulloblastoma, neuroblastoma, osteosarcoma, rhabdomyosarcoma, rhabdoid cancer, and nephroblastoma (Wilm's tumor). Other specific examples of cancer include, but are not limited to, carcinoma, lymphoma, blastoma (including medulloblastoma and retinoblastoma), sarcoma (including liposarcoma and synovial cell sarcoma), neuroendocrine tumors (including carcinoid tumors, gastrinoma, and islet cell cancer), mesothelioma, schwannoma (including acoustic neuroma), meningioma, adenocarcinoma, melanoma, and leukemia or lymphoid malignancies. More particular examples of such cancers include squamous cell cancer (e.g., epithelial squamous cell cancer), lung cancer including small-cell lung cancer (SCLC), non-small cell lung cancer (NSCLC), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer (including metastatic breast cancer), colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, Merkel cell cancer, mycoses fungoids, testicular cancer, esophageal cancer, tumors of the biliary tract, as well as head and neck cancer and hematological malignancies.

In some embodiments, the subject has melanoma. The melanoma may be at early stage or at late stage. In some embodiments, the subject has colorectal cancer. The colorectal cancer may be at early stage or at late stage. In some embodiments, the subject has non-small cell lung cancer. The non-small cell lung cancer may be at early stage or at late stage. In some embodiments, the subject has pancreatic cancer. The pancreatic cancer may be at early stage or late state. In some embodiments, the subject has a hematological malignancy. The hematological malignancy may be at early stage or late stage. In some embodiments, the subject has ovarian cancer. The ovarian cancer may be at early stage or at late stage. In some embodiments, the subject has breast cancer. The breast cancer may be at early stage or at late stage. In some embodiments, the subject has renal cell carcinoma. The renal cell carcinoma may be at early stage or at late stage.

In some embodiments, the cancer has elevated levels of T-cell infiltration.

The term “tumor,” as used herein, refers to all neoplastic cell growth and proliferation, whether malignant or benign, and all pre-cancerous and cancerous cells and tissues. The terms “cancer,” “cancerous,” and “tumor” are not mutually exclusive as referred to herein.

As used herein, the term “treatment” refers to clinical intervention designed to alter the natural course of the subject or cell being treated during the course of clinical pathology. Desirable effects of treatment include decreasing the rate of disease progression, ameliorating or palliating the disease state, and remission or improved prognosis. For example, a subject is successfully “treated” if one or more symptoms associated with cancer are mitigated or eliminated, including, but not limited to, reducing the proliferation of (or destroying) cancerous cells, decreasing symptoms resulting from the disease, increasing the quality of life of those suffering from the disease, decreasing the dose of other medications required to treat the disease, delaying the progression of the disease, and/or prolonging survival of subjects.

As used herein, “delaying progression of a disease” means to defer, hinder, slow, retard, stabilize, and/or postpone development of the disease (such as cancer). This delay can be of varying lengths of time, depending on the history of the disease and/or subject being treated. As is evident to one skilled in the art, a sufficient or significant delay can, in effect, encompass prevention, in that the subject does not develop the disease. For example, in a late stage cancer, such as development of metastasis, may be delayed.

An “effective amount” is at least the minimum concentration required to effect a measurable improvement or prevention of a particular disorder. An effective amount herein may vary according to factors such as the disease state, age, sex, and weight of the patient, and the ability of the antibody to elicit a desired response in the subject. An effective amount is also one in which any toxic or detrimental effects of the treatment are outweighed by the therapeutically beneficial effects. For prophylactic use, beneficial or desired results include results such as eliminating or reducing the risk, lessening the severity, or delaying the onset of the disease, including biochemical, histological and/or behavioral symptoms of the disease, its complications and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. In the case of cancer or tumor, an effective amount of the drug may have the effect in reducing the number of cancer cells; reducing the tumor size; inhibiting (i.e., slow to some extent or desirably stop) cancer cell infiltration into peripheral organs; inhibit (i.e., slow to some extent and desirably stop) tumor metastasis; inhibiting to some extent tumor growth; and/or relieving to some extent one or more of the symptoms associated with the disorder. An effective amount can be administered in one or more administrations. An effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

PD-1 axis antagonists are administered to a subject in conjunction with HPK1 antagonists to enhance an immune response or to treat cancer. As used herein, “in conjunction with” refers to administration of one treatment modality in addition to another treatment modality. As such, “in conjunction with” refers to administration of one treatment modality before, during, or after administration of the other treatment modality to the subject.

In some embodiments, the hpk1 and/or pd-1 antagonist is administered to the subject by administering a cell that expresses the hpk1 and/or pd-1 antagonist.

The PD-1 axis antagonist and the HPK1 antagonist may be administered in any suitable manner known in the art. For example, The PD-1 axis antagonist and the HPK1 antagonist may be administered sequentially (at different times) or concurrently (at the same time).

In some embodiments, the HPK1 antagonist is administered continuously. In other embodiments, the HPK1 antagonist is administered intermittently. In some embodiments, the PD-1 axis antagonist is administered continuously. In other embodiments, the PD-1 axis antagonist is administered intermittently. In some embodiments, the HPK1 antagonist is administered before administration of the PD-1 axis antagonist. In some embodiments, the HPK1 antagonist is administered simultaneously with administration of the PD-1 axis antagonist. In some embodiments, the HPK1 antagonist is administered after administration of the PD-1 axis antagonist. Moreover, treatment of a subject with an effective amount of a PD-1 axis antagonist and HPK1 antagonist can include a single treatment or can include a series of treatments.

The PD-1 axis antagonist and the HPK1 antagonist may be administered by the same route of administration or by different routes of administration. In some embodiments, the PD-1 axis antagonist is administered intravenously, intramuscularly, subcutaneously, topically, orally, transdermally, intraperitoneally, intraorbitally, by implantation, by inhalation, intrathecally, intraventricularly, intratumorally, or intranasally. In some embodiments, the HPK1 antagonist is administered intravenously, intramuscularly, subcutaneously, topically, orally, transdermally, intraperitoneally, intraorbitally, by implantation, by inhalation, intrathecally, intraventricularly, intratumorally, or intranasally.

It is understood that appropriate doses of such active compounds depends upon a number of factors within the knowledge of the ordinarily skilled physician or veterinarian. The dose(s) of the active compounds will vary, for example, depending upon the type of antagonist being administered, the age, body weight, general health, gender, and diet of the subject, the time of administration, the route of administration, the rate of excretion, any drug combination.

It will also be appreciated that the effective dosage of a PD-1 axis antagonist and HPK1 antagonist used for treatment may increase or decrease over the course of a particular treatment. Changes in dosage may result and become apparent from the results of diagnostic assays.

In some embodiments, the PD-1 axis antagonist and/or the HPK1 antagonist are administered to the subject at a dose of between about 0.001 μg/kg and about 1000 mg/kg, including but not limited to about 0.001 μg/kg, 0.01 μg/kg, 0.05 μg/kg, 0.1 μg/kg, 0.5 mg/kg, 1 μg/kg, 10 μg/kg, 25 μg/kg, 50 μg/kg, 100 mg/kg, 250 mg/kg, 500 μg/kg, 1 mg/kg, 5 mg/kg, 10 mg/kg, 25 mg/kg, 50 mg/kg, 100 mg/kg, and 200 mg/kg.

In some of the embodiments wherein the PD-1 axis antagonist is an antibody, the antibody is administered to the subject at a dose of between about 0.01 mg/kg and about 1000 mg/kg, including but not limited to about 0.01 mg/kg, 0.05 mg/kg, 0.1 mg/kg, 0.5 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 25 mg/kg, 50 mg/kg, 100 mg/kg, and 250 mg/kg.

In some embodiments, provided is a method for treating a cancer in a subject in need thereof comprising administering to the subject an effective amount of a PD-1 axis antagonist and a HPK1 antagonist, further comprising administering an additional therapy. The additional therapy may be radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, or a combination of the foregoing. The additional therapy may be in the form of adjuvant or neoadjuvant therapy. In some embodiments, the additional therapy is the administration of a small molecule enzymatic inhibitor or anti-metastatic agent. In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy is therapy targeting the PI3K/AKT/mTOR pathway, HSP90 inhibitor, tubulin inhibitor, apoptosis inhibitor, and/or chemopreventative agent. The additional therapy may be one or more of a chemotherapeutic agent.

A “chemotherapeutic agent” is a chemical compound useful in the treatment of cancer. Examples of chemotherapeutic agents include alkylating agents such as thiotepa and cyclophosphamide (CYTOXAN®); alkyl sulfonates such as busulfan, improsulfan, and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; pemetrexed; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; TLK-286; CDP323, an oral alpha-4 integrin inhibitor; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Nicolaou et al., Angew. Chem Intl. Ed. Engl., 33: 183-186 (1994)); dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®) and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, and floxuridine; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoids, e.g., paclitaxel (TAXOL®), albumin-engineered nanoparticle formulation of paclitaxel (ABRAXANE™), and doxetaxel (TAXOTERE®); chloranbucil; 6-thioguanine; mercaptopurine; methotrexate; platinum analogs such as cisplatin and carboplatin; vinblastine (VELBAN®); platinum; etoposide (VP-16); ifosfamide; mitoxantrone; vincristine (ONCOVIN®); oxaliplatin; leucovovin; vinorelbine (NAVELBINE®); novantrone; edatrexate; daunomycin; aminopterin; ibandronate; topoisomerase inhibitor RFS 2000; difluorometlhylornithine (DMFO); retinoids such as retinoic acid;

pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovovin.

Additional examples of chemotherapeutic agents include anti-hormonal agents that act to regulate, reduce, block, or inhibit the effects of hormones that can promote the growth of cancer, and are often in the form of systemic, or whole-body treatment. They may be hormones themselves. Examples include anti-estrogens and selective estrogen receptor modulators (SERMs), including, for example, tamoxifen (including NOLVADEX® tamoxifen), raloxifene (EVISTA®), droloxifene, 4-hydroxytamoxifen, trioxifene, keoxifene, LY117018, onapristone, and toremifene (FARESTON®); anti-progesterones; estrogen receptor down-regulators (ERDs); estrogen receptor antagonists such as fulvestrant (FASLODEX®); agents that function to suppress or shut down the ovaries, for example, leutinizing hormone-releasing hormone (LHRH) agonists such as leuprolide acetate (LUPRON® and ELIGARD®), goserelin acetate, buserelin acetate and tripterelin; anti-androgens such as flutamide, nilutamide and bicalutamide; and aromatase inhibitors that inhibit the enzyme aromatase, which regulates estrogen production in the adrenal glands, such as, for example, 4(5)-imidazoles, aminoglutethimide, megestrol acetate (MEGASE®), exemestane (AROMASIN®), formestanie, fadrozole, vorozole (RIVISOR®), letrozole (FEMARA®), and anastrozole (ARIMIDEX®). In addition, such definition of chemotherapeutic agents includes bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); as well as troxacitabine (a 1,3-dioxolane nucleoside cytosine analog); anti-sense oligonucleotides, particularly those that inhibit expression of genes in signaling pathways implicated in abherant cell proliferation, such as, for example, PKC-alpha, Raf, H-Ras, and epidermal growth factor receptor (EGF-R); vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); an anti-estrogen such as fulvestrant; EGFR inhibitor such as erlotinib or cetuximab; an anti-VEGF inhibitor such as bevacizumab; arinotecan; rmRH (e.g., ABARELIX®); 17AAG (geldanamycin derivative that is a heat shock protein (Hsp) 90 poison), and pharmaceutically acceptable salts, acids or derivatives of any of the above.

In some embodiments, the treatment results in a sustained response in the subject after cessation of the treatment. “Sustained response” refers to the sustained effect on reducing tumor growth after cessation of a treatment. For example, the tumor size may remain the same or smaller as compared to the size at the beginning of the administration phase. In some embodiments, the sustained response has a duration at least the same as the treatment duration, at least 1.5×, 2.0×, 2.5×, or 3.0× length of the treatment duration.

The treatment methods disclosed herein may result in a partial or complete response. As used herein, “complete response” or “CR” refers to disappearance of all target lesions; “partial response” or “PR” refers to at least a 30% decrease in the sum of the longest diameters (SLD) of target lesions, taking as reference the baseline SLD; and “stable disease” or “SD” refers to neither sufficient shrinkage of target lesions to qualify for PR, nor sufficient increase to qualify for PD, taking as reference the smallest SLD since the treatment started. As used herein, “overall response rate” (ORR) refers to the sum of complete response (CR) rate and partial response (PR) rate.

The treatment methods disclosed herein can lead to an increase in progression free survival and overall survival of the subject administered the PD-1 axis antagonist and HPK1 antagonist. As used herein, “progression free survival” (PFS) refers to the length of time during and after treatment during which the disease being treated (e.g., cancer) does not get worse. Progression-free survival may include the amount of time patients have experienced a complete response or a partial response, as well as the amount of time patients have experienced stable disease.

As used herein, “overall survival” refers to the percentage of subjects in a group who are likely to be alive after a particular duration of time.

In some embodiments, the subject that is administered a PD-1 axis antagonist and a HPK1 antagonist is a mammal, such as domesticated animals (e.g., cows, sheep, cats, dogs, and horses), primates (e.g., humans and non-human primates such as monkeys), rabbits, and rodents (e.g., mice and rats). In some embodiments, the subject treated is a human.

The subject in need of treatment for cancer may be a person demonstrating symptoms of cancer, one that has been diagnosed with cancer, a subject that is in remission from cancer, or a subject having an increased risk for developing cancer (e.g., a genetic predisposition, certain dietary or environmental exposures).

Particular embodiments of the subject matter described herein include the following:

1. A composition comprising a PD-1 axis antagonist and a HPK1 antagonist.

2. The composition of embodiment 1, wherein the PD-1 axis antagonist is selected from the group consisting of a PD-1 antagonist, a PD-L1 antagonist, and a PD-L2 antagonist.

3. The composition of embodiment 2, wherein the PD-1 axis antagonist is a PD-1 antagonist.

4. The composition of embodiment 3, wherein the PD-1 antagonist inhibits the binding of PD-1 to its ligand binding partners.

5. The composition of embodiment 4, wherein the PD-1 antagonist inhibits the binding of PD-1 to PD-L1.

6. The composition of embodiment 4, wherein the PD-1 antagonist inhibits the binding of PD-1 to PD-L2.

7. The composition of embodiment 4, wherein the PD-1 antagonist inhibits the binding of PD-1 to both PD-L1 and PD-L2.

8. The composition of any one of embodiments 4-7, wherein the PD-1 antagonist is an antibody.

9. The composition of embodiment 8, wherein the anti-PD-1 antibody is a monoclonal antibody.

10. The composition of embodiment 8, wherein the anti-PD-1 antibody is an antibody fragment selected from the group consisting of Fab, Fab′-SH, Fv, scFv, and (Fab′)₂ fragments.

11. The composition of any one of embodiments 8-10, wherein the anti-PD-1 antibody is a humanized antibody.

12. The composition of any one of embodiments 8-10, wherein the anti-PD-1 antibody is a human antibody.

13. The composition of embodiment 8, wherein the PD-1 antagonist is selected from the group consisting of:

a) MDX-1106;

b) Merck 3475;

c) CT-011;

d) an antibody that binds to an epitope capable of binding MDX-1106;

e) an antibody that binds to an epitope capable of binding Merck 3475;

f) an antibody that binds to an epitope capable of binding CT-011;

g) an antibody that competes with MDX-1106 for binding to PD-1 in a competitive binding assay;

h) an antibody that competes with Merck 3475 for binding to PD-1 in a competitive binding assay; and

i) an antibody that competes with CT-011 for binding to PD-1 in a competitive binding assay.

14. The composition of embodiment 8, wherein the PD-1 antagonist is MDX-1106.

15. The composition of embodiment 8, wherein the PD-1 antagonist is Merck 3475.

16. The composition of embodiment 8, wherein the PD-1 antagonist is CT-011.

17. The composition of embodiment 4, wherein the PD-1 antagonist is AMP-224.

18. The composition of embodiment 2, wherein the PD-1 axis antagonist is a PD-L1 antagonist.

19. The composition of embodiment 18, wherein the PD-L1 antagonist inhibits the binding of PD-L1 to PD-1.

20. The composition of embodiment 18, wherein the PD-L1 antagonist inhibits the binding of PD-L1 to B7-1.

21. The composition of embodiment 18, wherein the PD-L1 antagonist inhibits the binding of PD-L1 to both PD-1 and B7-1.

22. The composition of embodiment 18, wherein the PD-L1 antagonist is an antibody.

23. The composition of embodiment 22, wherein the anti-PD-L1 antibody is a monoclonal antibody.

24. The composition of embodiment 22, wherein the anti-PD-L1 antibody is an antibody fragment selected from the group consisting of Fab, Fab′-SH, Fv, scFv, and (Fab′)₂ fragments.

25. The composition of any one of embodiments 22-24, wherein the anti-PD-L1 antibody is a humanized antibody.

26. The composition of any one of embodiments 22-24, wherein the anti-PD-L1 antibody is a human antibody.

27. The composition of embodiment 22, wherein the PD-L1 antagonist is selected from the group consisting of:

a) YW243.55.S70;

b) MPDL3280A;

c) MEDI4736;

d) MDX-1105;

e) an antibody that binds to an epitope capable of binding YW243.55.S70;

f) an antibody that binds to an epitope capable of binding MPDL3280A;

g) an antibody that binds to an epitope capable of binding MEDI4736;

h) an antibody that binds to an epitope capable of binding MDX-1105;

i) an antibody that competes with YW243.55.S70 for binding to PD-1 in a competitive binding assay;

j) an antibody that competes with MPDL3280A for binding to PD-1 in a competitive binding assay;

k) an antibody that competes with MEDI4736 for binding to PD-1 in a competitive binding assay; and
1) an antibody that competes with MDX-1105 for binding to PD-1 in a competitive binding assay.

28. The composition of embodiment 22, wherein the PD-L1 antagonist is YW243.55.S70.

29. The composition of embodiment 22, wherein the PD-L1 antagonist is MPDL3280A.

30. The composition of embodiment 22, wherein the PD-L1 antagonist is MEDI4736.

31. The composition of embodiment 22, wherein the PD-L1 antagonist is MDX-1105.

32. The composition of embodiment 22, wherein the antibody comprises a heavy chain comprising HVR-H1 sequence of SEQ ID NO:35, HVR-H2 sequence of SEQ ID NO:36, and HVR-H3 sequence of SEQ ID NO:31; and a light chain comprising HVR-L1 sequence of SEQ ID NO:37, HVR-L2 sequence of SEQ ID NO:38, and HVR-L3 sequence of SEQ ID NO:39.

33. The composition of embodiment 22, wherein the antibody comprises a heavy chain variable region comprising the amino acid sequence of SEQ ID NO:27 and a light chain variable region comprising the amino acid sequence of SEQ ID NO:26.

34. The composition of embodiment 2, wherein the PD-1 axis antagonist is a PD-L2 antagonist.

35. The composition of embodiment 34, wherein the PD-L2 antagonist is an antibody.

36. The composition of embodiment 35, wherein the anti-PD-L2 antibody is a monoclonal antibody.

37. The composition of embodiment 34, wherein the PD-L2 antagonist is an immunoadhesin.

38. The composition of any one of embodiments 1-37, wherein the HPK1 antagonist is a specific HPK1 antagonist.

39. The composition of any one of embodiments 1-38, wherein the HPK1 antagonist is a competitive inhibitor.

40. The composition of embodiment 39, wherein the HPK1 antagonist is an ATP mimic.

41. The composition of any one of embodiments 1-40, wherein the composition further comprises a chemotherapeutic agent.

42. A pharmaceutical composition comprising the composition of any one of embodiments 1-41 and a pharmaceutically acceptable carrier.

43. A method for enhancing an immune response in a subject in need thereof, wherein the method comprises administering an effective amount of a combination of a PD-1 axis antagonist and a HPK1 antagonist.

44. The method of embodiment 43, wherein T cells in the subject have at least one of enhanced priming, enhanced activation, enhanced migration, enhanced proliferation, enhanced survival, and enhanced cytolytic activity relative to prior to the administration of the combination.

45. The method of embodiment 44, wherein the T cell activation is characterized by an elevated frequency of γ-IFN⁺ CD8 T cells or enhanced levels of IL-2 or granzyme B production by T cells relative to prior to administration of the combination.

46. The method of embodiment 45, wherein the number of T cells is elevated relative to prior to administration of the combination.

47. The method of any one of embodiments 44-46, wherein the T cell is an antigen-specific CD8 T cell.

48. The method of embodiment 43, wherein the antigen presenting cells in the subject have enhanced maturation and activation relative prior to the administration of the PD-1 axis antagonist and the HPK1 antagonist.

49. The method of embodiment 48, wherein the antigen presenting cells are dendritic cells.

50. The method of embodiment 48, wherein the maturation of the antigen presenting cells is characterized by increased frequency of CD83⁺ dendritic cells.

51. The method of embodiment 48, wherein the activation of the antigen presenting cells is characterized by elevated expression of CD80 and CD86 on dendritic cells.

52. The method of any one of embodiments 43-51, wherein the subject has cancer.

53. A method for treating cancer in a subject in need thereof, wherein the method comprises administering to the subject an effective amount of a combination of a PD-1 axis antagonist and a HPK1 antagonist.

54. The method of embodiment 52 or 53, wherein the cancer comprises at least one cancer selected from the group consisting of colorectal cancer, melanoma, non-small cell lung cancer, ovarian cancer, breast cancer, pancreatic cancer, a hematological malignancy, and a renal cell carcinoma; or the cancer is selected from the group consisting of carcinoma, lymphoma, blastoma (including medulloblastoma and retinoblastoma), sarcoma (including liposarcoma and synovial cell sarcoma), neuroendocrine tumors (including carcinoid tumors, gastrinoma, and islet cell cancer), mesothelioma, schwannoma (including acoustic neuroma), meningioma, adenocarcinoma, melanoma, and leukemia or lymphoid malignancies. More particular examples of such cancers include squamous cell cancer (e.g., epithelial squamous cell cancer), lung cancer including small-cell lung cancer (SCLC), non-small cell lung cancer (NSCLC), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer (including metastatic breast cancer), colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, Merkel cell cancer, mycoses fungoids, testicular cancer, esophageal cancer, tumors of the biliary tract, as well as head and neck cancer and hematological malignancies.

55. The method of any one of embodiments 52-54, wherein the cancer has elevated levels of T-cell infiltration.

56. The method of any one of embodiments 52-55, wherein the cancer cells in the subject selectively have elevated expression of MHC class I antigen expression relative to prior to the administration of the PD-1 axis antagonist and the HPK1 antagonist.

57. The method of any one of embodiments 52-56, wherein the method further comprises administering a chemotherapeutic agent to the subject.

58. The method of embodiment 57, wherein the chemotherapeutic agent is administered to the subject simultaneously with at least one of the PD-1 axis antagonist and the HPK1 antagonist.

59. The method of embodiment 57, wherein the chemotherapeutic agent is administered to the subject prior to administration of at least one of the PD-1 axis antagonist and the HPK1 antagonist.

60. The method of embodiment 57, wherein the chemotherapeutic agent is administered to the subject after administration of at least one of the PD-1 axis antagonist and the HPK1 antagonist.

61. The method of any one of embodiments 43-60, wherein the PD-1 axis antagonist is selected from the group consisting of a PD-1 antagonist, a PD-L1 antagonist, and a PD-L2 antagonist.

62. The method of embodiment 61, wherein the PD-1 axis antagonist is a PD-1 antagonist.

63. The method of embodiment 62, wherein the PD-1 antagonist inhibits the binding of PD-1 to its ligand binding partners.

64. The method of embodiment 63, wherein the PD-1 antagonist inhibits the binding of PD-1 to PD-L1.

65. The method of embodiment 63, wherein the PD-1 antagonist inhibits the binding of PD-1 to PD-L2.

66. The method of embodiment 63, wherein the PD-1 antagonist inhibits the binding of PD-1 to both PD-L1 and PD-L2.

67. The method of any one of embodiments 62-66, wherein the PD-1 antagonist is an antibody.

68. The method of embodiment 67, wherein the anti-PD-1 antibody is a monoclonal antibody.

69. The method of embodiment 67, wherein the anti-PD-1 antibody is an antibody fragment selected from the group consisting of Fab, Fab′-SH, Fv, scFv, and (Fab′)₂ fragments.

70. The method of embodiment 67 or 69, wherein the anti-PD-1 antibody is a humanized antibody.

71. The method of embodiment 67 or 69, wherein the anti-PD-1 antibody is a human antibody.

72. The method of embodiment 67, wherein the PD-1 antagonist is selected from the group consisting of:

a) MDX-1106;

b) Merck 3475;

c) CT-011;

d) an antibody that binds to an epitope capable of binding MDX-1106;

e) an antibody that binds to an epitope capable of binding Merck 3475;

f) an antibody that binds to an epitope capable of binding CT-011;

g) an antibody that competes with MDX-1106 for binding to PD-1 in a competitive binding assay;

h) an antibody that competes with Merck 3475 for binding to PD-1 in a competitive binding assay; and

i) an antibody that competes with CT-011 for binding to PD-1 in a competitive binding assay.

73. The method of embodiment 67, wherein the PD-1 antagonist is MDX-1106.

74. The method of embodiment 67, wherein the PD-1 antagonist is Merck 3475.

75. The method of embodiment 67, wherein the PD-1 antagonist is CT-011.

76. The method of embodiment 62, wherein the PD-1 antagonist is AMP-224.

77. The method of embodiment 61, wherein the PD-1 axis antagonist is a PD-L1 antagonist.

78. The method of embodiment 77, wherein the PD-L1 antagonist inhibits the binding of PD-L1 to PD-1.

79. The method of embodiment 77, wherein the PD-L1 antagonist inhibits the binding of PD-L1 to B7-1.

80. The method of embodiment 77, wherein the PD-L1 antagonist inhibits the binding of PD-L1 to both PD-1 and B7-1.

81. The method of embodiment 77, wherein the PD-L1 antagonist is an antibody.

82. The method of embodiment 81, wherein the anti-PD-L1 antibody is a monoclonal antibody.

83. The method of embodiment 81, wherein the anti-PD-L1 antibody is an antibody fragment selected from the group consisting of Fab, Fab′-SH, Fv, scFv, and (Fab′)₂ fragments.

84. The method of embodiment 81 or 82, wherein the anti-PD-L1 antibody is a humanized antibody.

85. The method of embodiment 81 or 82, wherein the anti-PD-L1 antibody is a human antibody.

86. The method of embodiment 81, wherein the PD-L1 antagonist is selected from the group consisting of:

a) YW243.55.S70;

b) MPDL3280A;

c) MEDI4736;

d) MDX-1105;

e) an antibody that binds to an epitope capable of binding YW243.55.S70;

f) an antibody that binds to an epitope capable of binding MPDL3280A;

g) an antibody that binds to an epitope capable of binding MEDI4736;

h) an antibody that binds to an epitope capable of binding MDX-1105;

i) an antibody that competes with YW243.55.S70 for binding to PD-1 in a competitive binding assay;

j) an antibody that competes with MPDL3280A for binding to PD-1 in a competitive binding assay;

k) an antibody that competes with MEDI4736 for binding to PD-1 in a competitive binding assay; and

l) an antibody that competes with MDX-1105 for binding to PD-1 in a competitive binding assay.

87. The method of embodiment 81, wherein the PD-L1 antagonist is YW243.55.S70.

88. The method of embodiment 81, wherein the PD-L1 antagonist is MPDL3280A.

89. The method of embodiment 81, wherein the PD-L1 antagonist is MEDI4736.

90. The method of embodiment 81, wherein the PD-L1 antagonist is MDX-1105.

91. The method of embodiment 81, wherein the antibody comprises a heavy chain comprising HVR-H1 sequence of SEQ ID NO:35, HVR-H2 sequence of SEQ ID NO:36, and HVR-H3 sequence of SEQ ID NO:31; and a light chain comprising HVR-L1 sequence of SEQ ID NO:37, HVR-L2 sequence of SEQ ID NO:38, and HVR-L3 sequence of SEQ ID NO:39.

92. The method of embodiment 81, wherein the antibody comprises a heavy chain variable region comprising the amino acid sequence of SEQ ID NO:27 and a light chain variable region comprising the amino acid sequence of SEQ ID NO:26.

93. The method of embodiment 61, wherein the PD-1 axis antagonist is a PD-L2 antagonist.

94. The method of embodiment 93, wherein the PD-L2 antagonist is an antibody.

95. The method of embodiment 94, wherein the anti-PD-L2 antibody is a monoclonal antibody.

96. The method of embodiment 93, wherein the PD-L2 antagonist is an immunoadhesin.

97. The method of any one of embodiments 43-96, wherein the HPK1 antagonist is a specific HPK1 antagonist.

98. The method of any one of embodiments 43-97, wherein the HPK1 antagonist is a competitive inhibitor.

99. The method of embodiment 98, wherein the HPK1 antagonist is an ATP mimic.

100. The method of any one of embodiments 43-99, wherein the treatment results in a sustained response in the subject after cessation of the treatment.

101. The method of any one of embodiments 43-100, wherein the PD-1 axis antagonist is administered intravenously, intramuscularly, subcutaneously, topically, orally, transdermally, intraperitoneally, intraorbitally, by implantation, by inhalation, intrathecally, intraventricularly, intratumorally, or intranasally.

102. The method of any one of embodiments 43-100, wherein the HPK1 antagonist is administered intravenously, intramuscularly, subcutaneously, topically, orally, transdermally, intraperitoneally, intraorbitally, by implantation, by inhalation, intrathecally, intraventricularly, intratumorally, or intranasally.

103. The method of any of embodiments 43-102, wherein the HPK1 antagonist is administered continuously.

104. The method of any of embodiments 43-102, wherein the HPK1 antagonist is administered intermittently.

105. The method of any of embodiments 43-102, wherein the HPK1 antagonist is administered before the PD-1 axis antagonist.

106. The method of any of embodiments 43-102, wherein the HPK1 antagonist is administered simultaneously with the PD-1 axis antagonist.

107. The method of any of embodiments 43-102, wherein the HPK1 antagonist is administered after the PD-1 axis antagonist.

108. A kit comprising a PD-1 axis antagonist and a package insert comprising instructions for using the PD-1 axis antagonist in combination with a HPK1 antagonist to enhance an immune response or treat cancer in a subject in need thereof

109. A kit comprising a HPK1 antagonist and a package insert comprising instructions for using the HPK1 antagonist in combination with a PD-1 axis antagonist to enhance an immune response or treat cancer in a subject in need thereof

110. A kit comprising a PD-1 axis antagonist, a HPK antagonist, and a package insert comprising instructions for using the PD-1 axis antagonist and the HPK antagonist to enhance an immune response or treat cancer in a subject in need thereof.

111. The kit of any one of embodiments 108-110, wherein the PD-1 axis antagonist is selected from the group consisting of a PD-1 antagonist, a PD-L1 antagonist, and a PD-L2 antagonist.

112. The kit of embodiment 111, wherein the PD-1 axis antagonist is a PD-1 antagonist.

113. The kit of embodiment 112, wherein the PD-1 antagonist inhibits the binding of PD-1 to its ligand binding partners.

114. The kit of embodiment 113, wherein the PD-1 antagonist inhibits the binding of PD-1 to PD-L1.

115. The kit of embodiment 113, wherein the PD-1 antagonist inhibits the binding of PD-1 to PD-L2.

116. The kit of embodiment 113, wherein the PD-1 antagonist inhibits the binding of PD-1 to both PD-L1 and PD-L2.

117. The kit of any one of embodiments 113-116, wherein the PD-1 antagonist is an antibody.

118. The kit of embodiment 117, wherein the anti-PD-1 antibody is a monoclonal antibody.

119. The kit of embodiment 117, wherein the anti-PD-1 antibody is an antibody fragment selected from the group consisting of Fab, Fab′-SH, Fv, scFv, and (Fab′)₂ fragments.

120. The kit of embodiment 117 or 118, wherein the anti-PD-1 antibody is a humanized antibody.

121. The kit of embodiment 117 or 118, wherein the anti-PD-1 antibody is a human antibody.

122. The kit of any one of embodiments 117-121, wherein the PD-1 antagonist is selected from the group consisting of:

a) MDX-1106;

b) Merck 3475;

c) CT-011;

d) an antibody that binds to an epitope capable of binding MDX-1106;

e) an antibody that binds to an epitope capable of binding Merck 3475;

f) an antibody that binds to an epitope capable of binding CT-011;

g) an antibody that competes with MDX-1106 for binding to PD-1 in a competitive binding assay;

h) an antibody that competes with Merck 3475 for binding to PD-1 in a competitive binding assay; and

i) an antibody that competes with CT-011 for binding to PD-1 in a competitive binding assay.

123. The kit of embodiment 117, wherein the PD-1 antagonist is MDX-1106.

124. The kit of embodiment 117, wherein the PD-1 antagonist is Merck 3475.

125. The kit of embodiment 117, wherein the PD-1 antagonist is CT-011.

126. The kit of embodiment 112, wherein the PD-1 antagonist is AMP-224.

127. The kit of embodiment 111, wherein the PD-1 axis antagonist is a PD-L1 antagonist.

128. The kit of embodiment 127, wherein the PD-L1 antagonist inhibits the binding of PD-L1 to PD-1.

129. The kit of embodiment 127, wherein the PD-L1 antagonist inhibits the binding of PD-L1 to B7-1.

130. The kit of embodiment 127, wherein the PD-L1 antagonist inhibits the binding of PD-L1 to both PD-1 and B7-1.

131. The kit of embodiment 127, wherein the PD-L1 antagonist is an antibody.

132. The kit of embodiment 131, wherein the anti-PD-L1 antibody is a monoclonal antibody.

133. The kit of embodiment 131, wherein the anti-PD-L1 antibody is an antibody fragment selected from the group consisting of Fab, Fab′-SH, Fv, scFv, and (Fab′)₂ fragments.

134. The kit of embodiment 131 or 132, wherein the anti-PD-L1 antibody is a humanized antibody.

135. The kit of embodiment 131 or 132, wherein the anti-PD-L1 antibody is a human antibody.

136. The kit of embodiment 131, wherein the PD-L1 antagonist is selected from the group consisting of:

a) YW243.55.S70;

b) MPDL3280A;

c) MEDI4736;

d) MDX-1105;

e) an antibody that binds to an epitope capable of binding YW243.55.S70;

f) an antibody that binds to an epitope capable of binding MPDL3280A;

g) an antibody that binds to an epitope capable of binding MEDI4736;

h) an antibody that binds to an epitope capable of binding MDX-1105;

i) an antibody that competes with YW243.55.S70 for binding to PD-1 in a competitive binding assay;

j) an antibody that competes with MPDL3280A for binding to PD-1 in a competitive binding assay;

k) an antibody that competes with MEDI4736 for binding to PD-1 in a competitive binding assay; and

l) an antibody that competes with MDX-1105 for binding to PD-1 in a competitive binding assay.

137. The kit of embodiment 131, wherein the PD-L1 antagonist is YW243.55.S70.

138. The kit of embodiment 131, wherein the PD-L1 antagonist is MPDL3280A.

139. The kit of embodiment 131, wherein the PD-L1 antagonist is MEDI4736.

140. The method of embodiment 131, wherein the PD-L1 antagonist is MDX-1105.

141. The kit of embodiment 131, wherein the antibody comprises a heavy chain comprising HVR-H1 sequence of SEQ ID NO:35, HVR-H2 sequence of SEQ ID NO:36, and HVR-H3 sequence of SEQ ID NO:31; and a light chain comprising HVR-L1 sequence of SEQ ID NO:37, HVR-L2 sequence of SEQ ID NO:38, and HVR-L3 sequence of SEQ ID NO:39.

142. The kit of embodiment 131, wherein the antibody comprises a heavy chain variable region comprising the amino acid sequence of SEQ ID NO:27 and a light chain variable region comprising the amino acid sequence of SEQ ID NO:26.

143. The kit of embodiment 111, wherein the PD-1 axis antagonist is a PD-L2 antagonist.

144. The kit of embodiment 143, wherein the PD-L2 antagonist is an antibody.

145. The kit of embodiment 144, wherein the anti-PD-L2 antibody is a monoclonal antibody.

146. The kit of embodiment 143, wherein the PD-L2 antagonist is an immunoadhesin.

147. The kit of any one of embodiments 108-146, wherein the HPK1 antagonist is a specific HPK1 antagonist.

148. The kit of any one of embodiments 108-146, wherein the HPK1 antagonist is a competitive inhibitor.

149. The kit of embodiment 148, wherein the HPK1 antagonist is an ATP mimic.

150. The kit of any one of embodiments 108-149, wherein the kit further comprises a chemotherapeutic agent.

151. The method of any of embodiments 43-102, wherein the HPK1 antagonist is a molecule which is capable of inhibiting the growth of MC38 tumor cells either as a single agent or in combination with a PD-1 or PD-L1 antagonist.

152. The method of any of embodiments 43-102, wherein the HPK1 antagonist is a molecule which in combination with a PD-1 or PD-L1 antagonist is capable of inhibiting the growth of MC38 tumor cells.

It is to be noted that the term “a” or “an” entity refers to one or more of that entity; for example, “a polypeptide” is understood to represent one or more polypeptides. As such, the terms “a” (or “an”), “one or more,” and “at least one” can be used interchangeably herein.

All technical and scientific terms used herein have the same meaning Efforts have been made to ensure accuracy with respect to numbers used (e.g. amounts, temperature, etc.) but some experimental errors and deviations should be accounted for.

Throughout this specification and the claims, the words “comprise,” “comprises,” and “comprising” are used in a non-exclusive sense, except where the context requires otherwise. It is understood that embodiments described herein include “consisting of” and/or “consisting essentially of” embodiments.

As used herein, the term “about,” when referring to a value is meant to encompass variations of, in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Where a range of values is provided, it is understood that each intervening value, to the tenth of the unit of the lower limit, unless the context clearly dictates otherwise, between the upper and lower limit of the range and any other stated or intervening value in that stated range, is encompassed within the invention. The upper and lower limits of these small ranges which may independently be included in the smaller rangers is also encompassed within the invention, subject to any specifically excluded limit in the stated range. Where the stated range includes one or both of the limits, ranges excluding either or both of those included limits are also included in the invention.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

The following examples are offered by way of illustration and not by way of limitation.

### EXAMPLES

### Example 1

### Anti-Tumor Effect of HPK1 Kinase Inhibition and PD-1 Blockade

### Methods:

HPK1 kinase-dead knock-in (HPK1.kd) mice were generated on a C57BL/6 background. Briefly, the point mutation K46E was introduced into the kinase domain of

HPK1, resulting in a kinase-inactive HPK1. Wild-type control mice were selected from within the HPK1.kd breeding colony and are therefore, littermate controls. Thirty-nine wild-type and thirty-seven HPK1.kd mice were inoculated with 1×10⁵ MC38 murine syngeneic colorectal tumor cells in HBSS:matrigel at a volume of 100 μL in the flank. Once tumors reached an average volume of 125-250 mm³, each cohort of wild-type and HPK1.kd mice were separated into two groups, and treated with either a control antibody (anti-gp120), or an anti-PDL1 antibody (clone 6E11.1.9, which is a murine anti-PDL1 antibody with the same CDRs as YW243.55.S70 and MPDL3280A). The therapeutic regimen consists of 10 mg/kg of anti-gp120 or anti-PDL1 antibody three times per week for three weeks, injected i.p. Nineteen wild-type mice and eighteen HPK1.kd mice were treated with anti-gp120, and twenty wild-type and nineteen HPK1.kd mice were treated with anti-PD-L1 antibody respectively. Tumor growth was monitored closely and measured twice weekly to determine if HPK1.kd mice had reduced tumor volumes relative to wild-type controls upon treatment with anti-PDL1 antibody. Animals with tumors that reached a volume of 2000 mm³ or greater or exceeded any IACUC Guidelines for Tumors in Rodents were euthanized or discussed with the veterinary staff.

### Results:

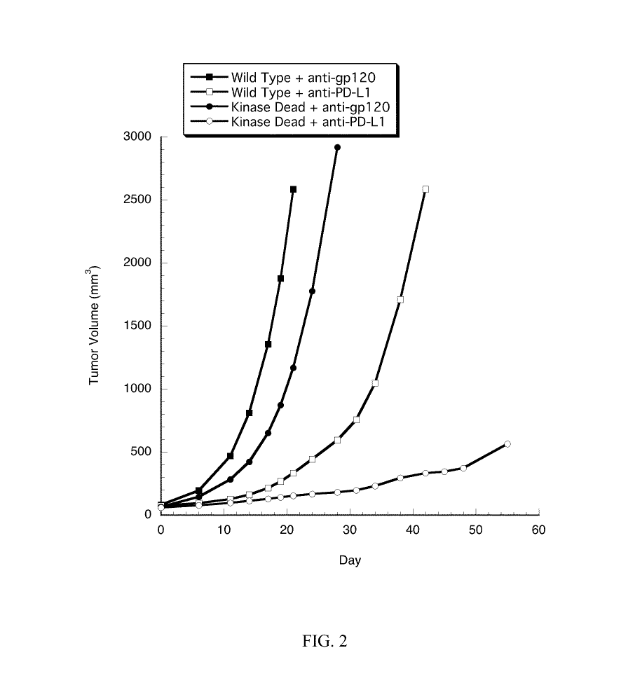

Prior to the initiation of anti-PDL1 treatment regimen, no dramatic differences in MC38 tumor take and growth measurements between the wild-type and HPK1.kd mice were observed (FIG. 2, day 0). Upon anti-PDL1 antibody treatment, MC38 tumor volumes in the HPK1.kd cohort show a significant more reduction than in wild-type mice, demonstrating an effective anti-tumor response of HPK1 kinase inhibition and PD-L1 co-blockade (FIG. 2).

### Example 2

### Anti-Tumor Effect of HPK1 Kinase Inhibition and PD1 Blockade

### Methods:

HPK1 kinase-dead knock-in (HPK1.kd) mice were generated on a C57BL/6 background. Briefly, the point mutation K46E was introduced into the kinase domain of HPK1, resulting in a kinase-inactive HPK1. Wild-type control mice were selected from within the HPK1.kd breeding colony and are therefore, littermate controls. Thirty wild-type and thirty HPK1.kd mice were inoculated with 1×10⁵ MC38 murine syngeneic colorectal tumor cells in HBSS:matrigel at a volume of 100 μL in the flank. Once tumors reached an average volume of 125-250 mm³, each cohort of wild-type and HPK1.kd mice was separated into groups of 15 mice, and treated with either a control antibody (anti-120), or an anti-PD1 antibody (clone 8F11.19.1.1, which is a murine anti-PD-1 antibody). The therapeutic regimen consists of 10 mg/kg of anti-gp120 or 5 mg/kg of anti-PD-1 antibody three times per week for three weeks, injected i.p. Tumor growth was monitored closely and measured twice weekly to determine if HPK1.kd mice had reduced tumor volumes relative to wild-type controls upon treatment with anti-PD-1 antibody. Animals with tumors that reached a volume of 2000 mm³ or greater or exceeded any IACUC Guidelines for Tumors in Rodents were euthanized or discussed with the veterinary staff.

### Results:

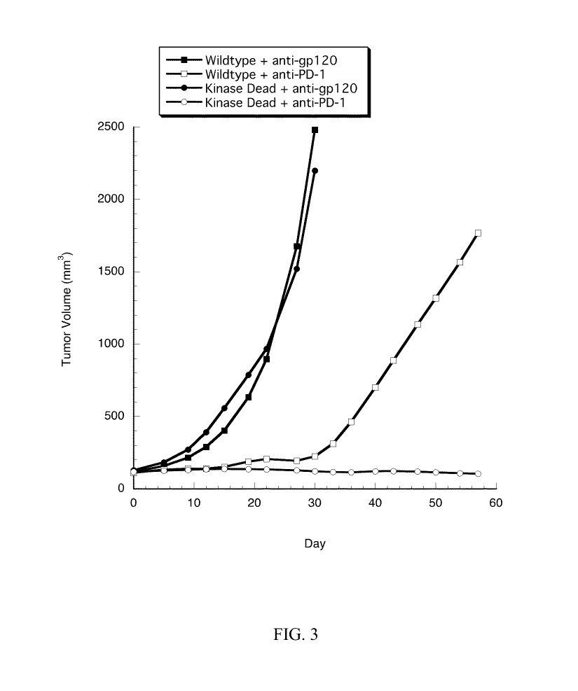

Prior to the initiation of anti-PD-1 treatment regimen, no dramatic differences in MC38 tumor take and growth measurements between the wild-type and HPK1.kd mice were observed (FIG. 3, day 0). Upon anti-PD-1 antibody treatment, MC38 tumor volumes in the HPK1.kd cohort remain flat during the course of the study and show more pronounced reduction than in wild-type mice, demonstrating an effective anti-tumor response of HPK1 kinase inhibition and PD-1 co-blockade (FIG. 3).

That which is claimed:

1. A method for treating cancer in a subject in need thereof, wherein the method comprises administering to the subject an effective amount of a combination of a PD-1 axis antagonist and a HPK1 antagonist.

2. The method of claim 1, wherein the cancer comprises at least one cancer selected from the group consisting of colorectal cancer, melanoma, non-small cell lung cancer, ovarian cancer, breast cancer, pancreatic cancer, a hematological malignancy, and a renal cell carcinoma.

3. The method of claim 1, wherein said cancer is selected from the group consisting of a carcinoma, lymphoma, blastoma, medulloblastoma, retinoblastoma, sarcoma, liposarcoma, synovial cell sarcoma, neuroendocrine tumors, carcinoid tumors, gastrinoma, islet cell cancer, mesothelioma, schwannoma, acoustic neuroma, meningioma, adenocarcinoma, melanoma, leukemia or lymphoid malignancies, squamous cell cancer, epithelial squamous cell cancer, lung cancer, small-cell lung cancer (SCLC), non-small cell lung cancer (NSCLC), adenocarcinoma of the lung, squamous carcinoma of the lung, cancer of the peritoneum, hepatocellular cancer, gastric or stomach cancer, gastrointestinal cancer, pancreatic cancer, glioblastoma, cervical cancer, ovarian cancer, liver cancer, bladder cancer, hepatoma, breast cancer, metastatic breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, Merkel cell cancer, mycoses fungoids, testicular cancer, esophageal cancer, tumors of the biliary tract, head and neck cancer, and hematological malignancies.

4. The method of claim 1, wherein the cancer has elevated levels of T-cell infiltration.

5. The method of claim 1, wherein said PD-1 axis antagonist is selected from the group consisting of a PD-1 antagonist, a PD-L1 antagonist, and a PD-L2 antagonist.

6. The method of claim 5, wherein the PD-1 axis antagonist is a PD-1 antagonist.

7. The method of any one of claim 6, wherein the PD-1 antagonist is an antibody.

8. The method of claim 7, wherein the anti-PD-1 antibody is a monoclonal antibody.

9. The method of claim 7, wherein the anti-PD-1 antibody is an antibody fragment selected from the group consisting of Fab, Fab′-SH, Fv, scFv, and (Fab′)₂ fragments.

10. The method of claim 7, wherein the PD-1 antagonist is MDX-1106.

11. The method of claim 7, wherein the PD-1 antagonist is Merck 3475.

12. The method of claim 7, wherein the PD-1 antagonist is CT-011.

13. The method of claim 6, wherein the PD-1 antagonist is AMP-224.

14. The method of claim 5, wherein the PD-1 axis antagonist is a PD-L1 antagonist.

15. The method of claim 14, wherein the PD-L1 antagonist is an antibody.

16. The method of claim 15, wherein the anti-PD-L1 antibody is a monoclonal antibody.

17. The method of claim 15, wherein the anti-PD-L1 antibody is an antibody fragment selected from the group consisting of Fab, Fab′-SH, Fv, scFv, and (Fab′)₂ fragments.

18. The method of claim 15, wherein the PD-L1 antagonist is YW243.55.S70.

19. The method of claim 15, wherein the PD-L1 antagonist is MPDL3280A.

20. The method of claim 15, wherein the PD-L1 antagonist is MEDI4736.

21. The method of claim 15, wherein the PD-L1 antagonist is MDX-1105.

22. The method of claim 1, wherein the PD-1 axis antagonist or the HPK1 antagonist is administered intravenously, intramuscularly, subcutaneously, topically, orally, transdermally, intraperitoneally, intraorbitally, by implantation, by inhalation, intrathecally, intraventricularly, intratumorally, or intranasally.

23. The method of claim 22, wherein the HPK1 antagonist is administered before the PD-1 axis antagonist.

24. The method of claim 22, wherein the HPK1 antagonist is administered simultaneously with the PD-1 axis antagonist.

25. The method of claim 22, wherein the HPK1 antagonist is administered after the PD-1 axis antagonist.
